(19) United States
(12) Patent Application Publication
(10) Pub. No.: US 2007/0099950 A1
(43) Pub. Date: May 3, 2007

(54) Pyridin-4-ylamine compounds useful in the treatment of neuropathic pain

(76) Inventors: Jongwon Lim, Lexington, MA (US); Julia K. Boueres, Pomezia (IT); Benito Munoz, Brookline, MA (US); Richard Pracitto, North Wales, PA (US); Nicholas Stock, San Diego, CA (US); Shankar Venkatraman, Lansdale, PA (US)

Correspondence Address: MERCK AND CO., INC, P O BOX 2000, RAHWAY, NJ, 07065-0907, US

(21) Appl. No.: 10/580,393

(22) PCT Filed: Nov. 18, 2004
(86) PCT No.: PCT/US04/38669

§ 371 Date: May 22, 2006

Related U.S. Application Data
(60) Provisional application No. 60/524,734, filed on Nov. 21, 2003.

(51) Int. Cl. A61K 31/473; C07D 219/12

(52) U.S. Cl. 514/297; 546/106

(57) ABSTRACT

The present invention is directed to a method of use of triazolo-pyridazine compounds in the treatment of neuropathic pain. The present invention is also directed to the use of triazolo-pyridazine compounds in the treatment of psychiatric and mood disorders such as, for example, schizophrenia, anxiety, depression, bipolar disorders, and panic, as well as in the treatment of pain, Parkinson's disease, cognitive dysfunction, epilepsy, circadian rhythm and sleep disorders—such as shift-work induced sleep disorder and jet-lag, drug addiction, drug abuse, drug withdrawal and other diseases. The present invention is also directed to novel triazolo-pyridazine compounds that selectively bind to α₂δ-1 subunit of Ca channels.

### BACKGROUND OF THE INVENTION

1. Field of the Invention

The present invention is directed pyridin-4-ylamine compounds and method of their use. In particular, this invention is directed to a method of use of pyridin-4-ylamine compounds in the treatment of neuropathic pain.

2. Related Background

A major mechanism in many physiological processes, including neurotransmission in the mammalian nervous system, is the opening and closing of voltage gated calcium channels (“VGCC”), also known as voltage sensitive calcium channels (“VSCC”). Such VGCC are formed by the assembly of subunit classes such as alpha 1 and alpha 2. One subunit in the alpha 2 class is the α₂δ subunit. The activity of the calcium channel can be modulated by the activities of the component subunits. For example, gabapentin is known to bind with high affinity to the α2δ subunit. Four isoforms of this α₂δ protein are known and gabapentin binds with high affinity to 2 of these (α₂δ-1 and α₂δ-2). The relative importance of these two activities in accounting for the efficacy and adverse effects of gabapentin is not known. Compounds that display high-affinity binding to the α₂δ subunit of voltage gated calcium channels have been shown to be efficacious for the treatment of, for example, neuropathic pain. See, J. Biol. Chem., 271(10):5768-5776(1996) and J. Med. Chem., 41:1838-1845(1998). Nonetheless, if one isoform is more controlling of the channel modulation, while the other is less, then compounds that are selective to the controlling isoform are likely to be more efficacious and display fewer side-effects.

Thus, it is desirable to identify other compounds that display high-affinity binding to the α₂δ subunit of voltage gated calcium channels to provide new medicines in the treatment of neuropathic pain. Further, such compounds can be useful in the treatment of psychiatric and mood disorders such as, for example, schizophrenia, anxiety, depression, bipolar disorders, and panic, as well as in the treatment of pain, Parkinson's disease, cognitive dysfunction, epilepsy, circadian rhythm and sleep disorders—such as shift-work induced sleep disorder and jet-lag, drug addiction, drug abuse, drug withdrawal and other diseases.

International Patent Publication No. WO 01/88101 describes a cell line for the expression of an α₂δ2 calcium channel subunit.

6-Methyl-6H-pyrrolo[3,4-d]pyridazine is described in MM.J. Duflos et al., Tetrahedron Lett., 3453-3454(1973). 1,4,5,7-tetramethyl-6-phenyl-6H-pyrrolo[3,4-d]pyridazine, 1,4,5-trimethyl-6,7-diphenyl-6H-pyrrolo[3,4-d]pyridazine, 5,7-dimethyl-1,4,6-triphenyl-6H-pyrrolo[3,4-d]pyridazine, 5-methyl-1,4,6,7-tetraphenyl-6H-pyrrolo[3,4-d]pyridazine, 1,4-bis-(4-methoxy-phenyl)-5,7-dimethyl-6-phenyl-6H-pyrrolo[3,4-d]pyridazine, 1,4-bis-(4-methoxy-phenyl)-5-methyl-6,7-diphenyl-6H-pyrrolo[3,4-d]pyridazine, and 1,4-diethyl-5,7-dimethyl-6-phenyl-6H-pyrrolo[3,4-d]pyridazine are described in R. Rips et al., J. Org. Chem., 24:551-554(1959). 1,4,5,7-Tetramethyl-6H-pyrrolo[3,4-d]pyridazine, N-(1,4,5,7-tetramethyl-pyrrolo[3,4-d]pyridazin-6-yl)-benzamide, 1,4,5,7-tetramethyl-pyrrolo[3,4-d]pyridazin-6-ylamine picrate, and 1,4,5,7-tetramethyl-pyrrolo[3,4-d]pyridazin-6-ylamine are described in W. L. Mosby, J. Chem. Soc., 3997-4003(1957). 5,7-Dimethyl-6-phenyl-6H-pyrrolo[3,4-d]pyridazine is described in R. Rips et al., J. Org. Chem., 24:372-374(1959).

5,7-Dimethyl-2-phenacyl-6H-pyrrolo[3,4-d]pyridazinium bromide (also known as 5,7-dimethyl-2-(2-oxo-2-phenyl-ethyl)-6H-pyrrolo[3,4-d]pyridazin-2-ium bromide) and 2-(2-methoxycarbonylvinyl)-5,7-dimethyl-6H-pyrrolo[3,4-d]pyridazinium tetrafloroborate are described in F. Fuentes-Rodriguez et al., J. Chem. Res. Miniprint, 11:2901-2914(1987). 5,7-Diphenyl-6H-pyrrolo[3,4-d]pyridazine is described in T. Hernandez et al., J. Chem. Soc., Perkins Trans., 1:899-902(1985), and F. F. Rodriguez et al., J. Chem. Res. Miniprint, 11:3001-3001(1987). 5,6,7-Trimethyl-6H-pyrrolo[3,4-d]pyridazine is described in T. Hernandez et al., J. Chem. Soc., Perkin Trans., 1:899-902(1985), F. Fuentes-Rodriguez et al., J. Chem. Res. Miniprint, 11:2901-2914(1987), and R. von Kreher et al., Agnew Chem., 82:958(1970).

1,4-Diphenyl-7,8,9,10-tetrahydro-pyridazino[4,5-a]indolizine (also known as 1,4-diphenyl-5,6,7,8-tetrahydro-2,3,8a-triaza-fluorene) and 5-methyl-1,4-diphenyl-7,8,9,10-tetrahydro-pyridazino[4,5-a]indolizine (also known as 9-methyl-1,4-diphenyl-5,6,7,8-tetrahydro-2,3,8a-triaza-fluorene) are described in T. Uchida et al., J. Heterocycl. Chem., 15: 1303-1307(1978). 6-Benzyl-1,4-diphenyl-5-p-tolyl-6H-pyrrolo[3,4-d]pyridazine, 6-benzyl-5-(2-chloro-phenyl)-1,4-diphenyl-6H-pyrrolo[3,4-d]pyridazine, 1,4,5,6,7-pentaphenyl-6H-pyrrolo[3,4-d]pyridazine, 6,7,10,11-tetraphenyl-pyridazino[4′,5′:3,4]pyrrolo[1,2-a]quinoxaline (also known as 6,7,10,11-tetraphenyl-5,8,9,11a-tetraaza-benzo[a]fluorene), 11-(4-nitro-phenyl)-6,7,10-triphenyl-pyridazino[4′,5′:3,4]pyrrolo[1,2-a]quinoxaline (also known as 11-(4-nitro-phenyl)6,7,10-triphenyl-5,8,9, 11a-tetraaza-benzo[a]fluorene), and 6-benzyl-1,4,5-triphenyl-6H-pyrrolo[3,4-d]pyridazine are described in T. Uchida et al., J. Heterocycl. Chem., 15:241-248(1978).

9,12-Diphenyl-pyridazino[4′,5′:3,4]pyrrolo[2,1-a]isoquinoline, 5-methylsulfanyl-1,4,6,7-tetraphenyl-6H-pyrrolo[3,4-d]pyridazine, and 1,4,6,7-tetraphenyl-6H-pyrrolo[3,4-d]pyridazine-5-carboxylic acid ethyl ester are described in K. T. Potts et al., J. Org. Chem., 42:1639-1644(1977). 7,10-Diphenyl-pyridazino[4′,5′:3,4]pyrrolo[1,2-a]quinoline, and 11,14-diphenyl-pyridazino[4′,5′:3,4]pyrrolo[1,2-f]phenanthridine (also known as 9,12-diphenyl-10,11,13a-triaza-indeno[1,2-l]phenanthrene) are described in K. T. Potts et al., J. Org. Chem., 44:977-979(1979).

1-Oxo-7-oxy-6b,11b-dihydro(pyridazino[4′,5′-c]-pyrrolo)[2.1-c]benzoxazine-1,4 (also known as 11-hydroxy-5-oxa-8,9,11a-triaza-benzo[a]fluoren-6-one) is described in Kumashiro et al., Nippon Kagaku Zasshi., 82:1072-1074(1961). 10-Methyl-1,4-diphenyl-8,9-dihydro-7H-benzo(ef)pyridazino[4,5-a]cycl[3.3.2]azine, and 11-methyl-1,4-diphenyl-7,8,9,10-tetrahydrocyclohepta(ef)pyridazino[4,5-a]cycl[3.3.2]azine are described in M. Noguchi et al., J. Heterocycl. Chem., 22:1049-1053(1985).

1,4-Dichloro-5,6,7-trimethyl-6H-pyrrolo[3,4-d]pyridazine, 1-chloro4-ethoxy-5,6,7-triethyl-6H-pyrrolo[3,4-d]pyridazine, 1-chloro-5,6,7-trimethyl-6H-pyrrolo[3,4-d]pyridazinium chloride 1-ethoxy-2,5,6,7-tetramethyl-6H-pyrrolo[3,4-d]pyridazinium tetrafluoroborate, 1-ethoxy-5,6,7-trimethyl-2H,6H-pyrrolo[3,4-d]pyridazinium tetrafluoroborate, 1-ethoxy-3-ethyl-5,6,7-trimethyl-6H-pyrrolo[3,4-d]pyridazinium tetrafluoroborate, and 1-ethoxy-5,6,7-trimethyl-6H-pyrrolo[3,4-d]pyridazine are described in S. Inel et al., Tetrahedron, 40:3979-3986(1984).

5-Cyano-1,4-dimethylpyridazino[4,5-a]indolizine (also known as 1,4-dimethyl-2,3,8a-triaza-fluorene-9-carbonitrile), 1,4-dimethyl-6-phenyl-2,3,8a-triaza-fluorene-9-carbonitrile, 6-benzolyl-1,4-dimethyl-2,3,8a-triaza-fluorene-9-carbonitrile, 6-benzyl-1,4-diphenyl-2,3,8a-triaza-fluorene-9-carbonitrile, and 1,4,6-trimethyl-2,3,8a-triaza-fluorene-9-carbonitrile are described in K. Matsumoto et al., J. Heterocycl. Chem., 25:1793-1801(1988). 5-Cyano-1,4-dimethylpyridazino[4,5-a]indolizine (also known as 1,4-diphenyl-2,3,8a-triaza-fluorene-9-carbonitrile) is described in K. Matsumoto et al., J. Heterocycl. Chem, 25:1793-1801(1988), and K. Matsumoto et al., Heterocycles, 20:1525-1529(1983). 6-Methyl-1,4diphenyl-2,3,8a-triaza-fluorene-9-carbonitrile, 6-benzoyl-1,4-diphenyl-2,3,8a-triaza-fluorene-9-carbonitrile, and 1,4,6-triphenyl-2,3,8a-triaza-fluorene-9-carbonitrile are described in K. Matsumoto et al., J. Heterocycl. Chem., 25:1793-1801(1988), K. Matsumoto et al., Heterocycles, 34:2239-2242(1992), K. Matsumoto et al., Heterocycles, 20:1525-1529(1983), and K. Matsumoto et al., Can. J. Chem., 71:529-533(1993). 5,7-Dimethyl-1,4-diphenyl-2,3,8a-triaza-fluorene-9-carbonitrile, and 9,12-diphenyl-pyridazino[4′,5′:3,4]pyrrolo[2,1-a]isoquinoline-8-carbonitrile are described in K. Matsumoto et al., Heterocycles, 34:2239-2242(1992), and K. Matsumoto et al., Cam. J. Chem., 71:529-533(1993).

Dimethyl 3,12,13,17-tetramethyl-7²,7³-diazabenzo[g]porphyrin-2,18-dipropionate is described in I. A. Chaudhry et al., Aust. J. Chem., 35:1185-11201(1982). 5,6-Dihydro-2,3-dimethoxypyridazino[4′,5′:3,4]pyrrolo[2,1-a]isochinolin-9-ol, 5,6-dihydro-2,3-dimethoxypyridazino[4′,5′:3,4]pyrrolo[2,1-a]isochinolin-9-ol-hydrochloride, and 3-methyl-6,9-diphenylthiazolo[3′,2′:1,2]pyrrolo[3,4-d]pyridine (also known as 1-methyl-4,7-diphenyl-3-thia-5,6,8a-triaza-cyclopenta[a]indene) are described in W. Losel et al., Chem. Ber., 118:413-427 (1985). 1,4-Diphenylpyridazino[4′,5′:3,4]pyrrolo[2,1-b]benzothiazole (also known as 1,4-diphenyl-5-thia-2,3,9b-triaza-indeno[2,1-a]indene) is described in N. Abe et al., Bull. Chem. Soc. Japan, 55:200-203(1982).

Nevertheless, there is a need to identify pyridin-4-ylamine compounds that display high-affinity binding—particularly selective binding—to the α₂δ subunit of voltage gated calcium channels to provide new medicines in the treatment of neuropathic pain, as well as psychiatric and mood disorders such as, for example, schizophrenia, anxiety, depression, bipolar disorders, and panic, as well as in the treatment of pain, Parkinson's disease, cognitive dysfunction, epilepsy, circadian rhythm and sleep disorders—such as shift-work induced sleep disorder and jet-lag, drug addiction, drug abuse, drug withdrawal and other diseases.

### SUMMARY OF THE INVENTION

The present invention is directed to a method of use of pyridin-4-ylamine compounds in the treatment of neuropathic pain. The present invention is also directed to the use of pyridin-4-ylamine compounds in the treatment of psychiatric and mood disorders such as, for example, schizophrenia, anxiety, depression, bipolar disorders, and panic, as well as in the treatment of pain, Parkinson's disease, cognitive dysfunction, epilepsy, circadian rhythm and sleep disorders—such as shift-work induced sleep disorder and jet-lag, drug addiction, drug abuse, drug withdrawal and other diseases. The present invention is also directed to novel pyridin-4-ylamine compounds that selectively bind to α₂δ-1 subunit of Ca channels.

### DETAILED DESCRIPTION OF THE INVENTION

In one aspect the invention is directed to compounds of Formula (I): 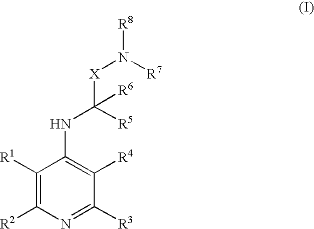
or N-oxide and pharmaceutically acceptable salts thereof, wherein
R¹ is selected from the group consisting of
   (a) Hydrogen,
   (b) halo,
   (c) —C₀₋₆alkyl-aryl,
   (d) —C₀₋₆alkyl-heteroaryl,
   (e) —C₁₋₆alkyl, optionally substituted with 1, 2 or 3 halo atoms,
   (f) —C₀₋₆alkyl-C₃₋₆cycloalkyl, and
   (g) -heteroC₀₋₆alkyl;
R² is selected from the group consisting of
   (a) Hydrogen,
   (b) halo,
   (c) —C₀₋₆alkyl-aryl,
   (d) —C₀₋₆alkyl-heteroaryl,
   (e) —C₁₋₆alkyl, optionally substituted with 1, 2 or 3 halo atoms,
   (f) —C₀₋₆alkyl-C₃₋₆cycloalkyl, and
   (g) -heteroC₀₋₆alkyl;
or R¹ and R² are joined so that together with the atoms to which they are attached there is formed a saturated or unsaturated ring with 0-4 heteroatoms, selected from phenyl, said ring optionally mono or di-substituted with sustituents independently selected from hydroxyl, halo, —C₁₋₆alkyl, —O—C₁₋₆alkyl, —NO₂, —C₃, aryl, heteroaryl, and heteroC₁₋₆alkyl;
R³ is selected from the group consisting of
   (a) Hydrogen,
   (b) halo,
   (c) —C₀₋₆alkyl-aryl,
   (d) —C₀₋₆alkyl-heteroaryl,
   (e) —C₁₋₆alkyl, optionally substituted with 1, 2 or 3 halo atoms,
   (f) —C₀₋₆alkyl-C₃₋₆cycloalkyl, and
   (g) -heteroC₀₋₆alkyl;
R⁴ is selected from the group consisting of
   (a) Hydrogen,
   (b) halo,
   (c) —C₀₋₆alkyl-aryl,
   (d) —C₀₋₆alkyl-heteroaryl,
   (e) —C₁₋₆alkyl, optionally substituted with 1, 2 or 3 halo atoms,
   (f) —C₀₋₆alkyl-C₃₋₆cycloalkyl, and
   (g) -heteroC₀₋₆alkyl;
      or R³ and R⁴ are joined so that together with the atoms to which they are attached there is formed a saturated or unsaturated ring with 0-4 heteroatoms, selected from phenyl, said ring optionally mono or di-substituted with sustituents independently selected from hydroxyl, halo, —C₁₋₆alkyl, —O—C₁₋₆alkyl, —NO₂, —CF₃, aryl, heteroaryl, and heteroC₁₋₆alkyl;
R⁵ is selected from the group consisting of
   (a) Hydrogen,
   (b) —C₀₋₆alkyl-aryl,
   (c) —C₀₋₆alkyl-heteroaryl,
   (d) —C₁₋₆alkyl, optionally substituted with 1, 2 or 3 halo atoms,
   (e) —C₀₋₆alkyl-C₃₋₆cycloalkyl, and
   (f) -heteroC₀₋₆allyl;
      wherein R⁵ choices (b), (c), (d), (e) and (f) are each optionally substituted with a substituent selected from hydroxyl, halo, —NO₂ and CF₃;
R⁶ is selected from the group consisting of
   (a) hydrogen,
   (b) —C₁₋₃alkyl,
      wherein R⁶ choices (b) is optionally substituted with a substituent selected from hydroxyl, halo, —NO₂ and CF₃;
      or R⁵ and R⁶ are joined so that together with the atoms to which they are attached there is formed a saturated or unsaturated ring with 04 heteroatoms, selected from phenyl, said ring optionally mono or di-substituted with sustituents independently selected from hydroxyl, halo, —C₁₋₆alkyl, —O—C₁₋₆alkyl, —NO₂, —CF₃, aryl, heteroaryl, and heteroC₁₋₆alkyl;
R⁷ is selected from the group consisting of
   (a) Hydrogen,
   (b) —C₀₋₃alkyl-aryl,
   (c) —C₀₋₃alkyl-heteroaryl,
   (d) —C₁₋₆alkyl,
   (e) —C₀₋₃alkyl-C₃₋₆cycloalkyl, and
   (f) -heteroC₀₋₆alkyl;
      wherein R⁷ choices (b), (c), (d), (e) and (f) are each optionally substituted with a substituent selected from hydroxyl, halo, —NO₂ and CF₃;
R⁸ is selected from the group consisting of
   (a) Hydrogen,
   (b) —C₀₋₃alkyl-aryl,
   (c) —C₀₋₃alkyl-heteroaryl,
   (d) —C₁₋₆alkyl,
   (e) —C₀₋₃alkyl—C₃₋₆cycloalkyl, and
   (f) -heteroC₀₋₆alkyl;
      wherein R⁸ choices (b), (c), (d), (e) and (f) are each optionally substituted with a substituent selected from hydroxyl, halo, —NO₂ and CF₃;
      or R⁶ and R⁸ are joined so that together with the atoms to which they are attached there is formed a saturated or unsaturated ring with 1-4 heteroatoms, selected from phenyl, said ring optionally mono or di-substituted with sustituents independently selected from hydroxyl, halo, —C₁₋₆alkyl, —O—-C₁₋₆alkyl, —NO₂, —CF₃, aryl, heteroaryl, and heteroC₁₋₆alkyl;
      or R⁷ and R⁸ are joined so that together with the atoms to which they are attached there is formed a saturated or unsaturated ring with 04 heteroatoms, selected from phenyl, said ring optionally mono or di-substituted with sustituents independently selected from hydroxyl, halo, —C₁₋₆alkyl, —O—C₁₋₆alkyl, —NO₂, —CF₃, aryl, heteroaryl, and heteroC₁₋₆alkyl;
R⁹ is selected from the group consisting of
   (a) C₁₋₆alkyl,
   (b) C₃₋₆cycloalkyl,
   (c) aryl, and
   (d) heteroaryl; and
X is selected from the group consisting of
   (a) C₁₋₆alkylene,
   (b) O,
   (c) S,
   (d)S(O)₂,
   (e) NR⁹, and
   (f) C(O),
      with the proviso that either R¹ and R² or R³ and R⁴ must be joined together to form a ring.

Within this embodiment there is a genus wherein:
R¹ is selected from the group consisting of
   (a) hydrogen,
   (b) phenyl or naphthyl,
   (c) —C₁₋₆alkyl, optionally substituted with 1, 2 or 3 halo atoms,
   (d) —O—C₁₋₆alkyl; and
R² is selected from the group consisting of
   (a) hydrogen,
   (b) phenyl or naphthyl,
   (c) —C₁₋₆alkyl, optionally substituted with 1, 2 or 3 halo atoms
   (d) —O—C₁₋₆alkyl;
      or R¹ and R² are joined so that together with the atoms to which they are attached there is formed a ring selected from phenyl, naphthyl and cyclohexyl, said ring optionally mono or di-substituted with sustituents independently selected from hydroxyl, halo, —C₁₋₆alkyl, —O—C₁₋₆alkyl, —NO₂ and —CF₃.

Within this genus, there is a sub-genus wherein:

R¹ and R² are joined so that together with the atoms to which they are attached there is formed a ring selected from phenyl, naphthyl and cyclohexyl, said ring optionally mono or di-substituted with sustituents independently selected from hydroxyl, halo, —C₁₋₆alkyl, —O—C₁₋₆alkyl, —NO₂ and —CF₃.

Within this embodiment there is another genus wherein:
R³ is selected from the group consisting of
   (a) hydrogen,
   (b) phenyl or naphthyl,
   (c) —C₁₋₆alkyl, optionally substituted with 1, 2 or 3 halo atoms
   (d) —O—C₁₋₆alkyl; and
R⁴ is selected from the group consisting of
   (a) hydrogen,
   (b) phenyl, naphthyl or pyridyl,
   (c) —C₁₋₆alkyl, optionally substituted with 1, 2 or 3 halo atoms,
   (d) —O—C₁₋₆alkyl;
      or R³ and R⁴ are joined so that together with the atoms to which they are attached there is formed a ring selected from phenyl and cyclohexyl, said ring optionally mono or di-substituted with sustituents independently selected from hydroxyl, halo, —C₁₋₆alkyl, —O—C₁₋₆alkyl, —NO₂ and —CF₃.

Within this genus, there is a subgenus wherein:

R³ and R⁴ are joined so that together with the atoms to which they are attached there is formed a ring selected from phenyl and cyclohexyl, said ring optionally mono or di-substituted with sustituents independently selected from hydroxyl, halo, —C₁₋₆alkyl, —O—C₁₋₆alkyl, —NO₂ and —CF₃.

Within this embodiment there is another genus wherein:
R⁵ is selected from the group consisting of
   (a) hydrogen,
   (b) —C₁₋₃alkyl;
   (c) phenyl or naphthyl,
   (d) C₃₋₆cycloalkyl.

Within this embodiment there is another genus wherein:
R⁶ is selected from the group consisting of
   (a) hydrogen,
   (b) —C₁₋₃alkyl.

Within this embodiment there is another genus wherein:
R⁷ is selected from the group consisting of
   (a) hydrogen,
   (b) —C₁₋₆alkyl,
   (c) —C₁₋₄alkylphenyl.

Within this embodiment, there is a genus wherein:
R⁸ is selected from the group consisting of
   (a) hydrogen,
   (b) —C₁₋₆alkyl.

Within this embodiment there is another genus wherein:
X is CH₂CH₂CH₂.

Within this embodiment, there is a genus of compounds of Formula II: 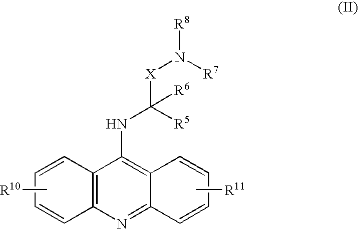
wherein:
R⁵ is selected from the group consisting of
   (a) hydrogen,
   (b) —C₁₋₃alkyl,
   (c) phenyl or naphthyl,
   (d) —C₃₋₆cycloalkyl;
R⁶ is
   (a) hydrogen,
   (b) —C₁₋₃alkyl;
R⁷ is selected from the group consisting of
   (a) hydrogen,
   (b) —C₁₋₄alkyl,
   (c) —C₁₋₂alkylphenyl;
R⁸ is —C₁₋₄alkyl;
R¹⁰ and R¹¹ are each selected from the group consisting of Hydrogen, hydroxyl, halo, —C₁₋₃alkyl, —O—C₁₋₃alkyl, —NO₂ and —CF₃; and X is CH2CH2CH2.

Within this genus there is a sub-genus wherein:

R6 is hydrogen.

Within this subgenus, there is a class wherein:

R⁵ is selected from the group consisting of —C₁₋₃alkyl, phenyl, naphthyl and —C₃₋₆cycloalkyl.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, indenyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like.

The term “aryl” means an aromatic substituent which is a single ring or multiple rings fused together. When formed of multiple rings, at least one of the constituent rings is aromatic. The preferred aryl substituents are phenyl and naphthyl groups.

The term “cycloalkyloxy” unless specifically stated otherwise includes a cycloalkyl group connected by a short C₁₋₂alkyl length to the oxy connecting atom.

The term “C₀₋₆alkyl” includes alkyls containing 6, 5, 4, 3, 2, 1, or no carbon atoms. An alkyl with no carbon atoms is a hydrogen atom substituent when the alkyl is a terminal group and is a direct bond when the alkyl is a bridging group.

The term “hetero” unless specifically stated otherwise includes one or more O, S, or N atoms. For example, heterocycloalkyl, and heteroaryl include ring systems that contain one or more O, S, or N atoms in the ring, including mixtures of such atoms. The hetero atoms replace ring carbon atoms. Thus, for example, a heterocycloC₅alkyl is a five-member ring containing from 4 to no carbon atoms. Examples of heteroaryls include pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, benzoxazolyl, isoxazolyl, thiazolyl, benzothiazolyl, isothiazolyl, imidazolyl, benzimidazolyl, oxadiazolyl, thiadiazolyl, triazolyl, and tetrazolyl. Examples of heterocycloalkyls include azetidinyl, pyrrolidinyl, piperidinyl, piperazinyl, morpholinyl, tetrahydrofuranyl, imidazolinyl, pyrolidin-2-one, piperidin-2-one, and thiomorpholinyl.

The term “heteroC₀₋₄alkyl” means a heteroalkyl containing 3, 2, 1, or no carbon atoms. However, at least one heteroatom must be present. Thus, as an example, a heteroC₀₋₄alkyl having no carbon atoms but one N atom would be a —NH— if a bridging group and a —NH₂ if a terminal group. Analogous bridging or terminal groups are clear for an O or S heteroatom.

The term “amine” unless specifically stated otherwise includes primary, secondary and tertiary amines substituted with C₀₋₆alkyl.

The term “carbonyl” unless specifically stated otherwise includes a C₀₋₆alkyl substituent group when the carbonyl is terminal.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. Further, optionally substituted multiple moieties such as, for example, alkylaryl are intended to mean that the aryl and the alkyl groups are optionally substituted. If only one of the multiple moieties is optionally substituted then it will be specifically recited such as “an alkylaryl, the aryl optionally substituted with halogen or hydroxyl.”

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes the use of all such possible diastereomers as well as their racemic mixtures, their substantially pure resolved enantiomers, all possible geometric isomers, and pharmaceutically acceptable salts thereof. The above Formula I is shown without a definitive stereochemistry at certain positions. The present invention includes the use of all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “pharmaceutically acceptable salts” refers to salts prepared from pharmaceutically acceptable non-toxic bases or acids. When the compound used in the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound used in the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

The pharmaceutical compositions used of pyridin-4-ylamine compounds of the present invention comprise a compound represented by Formula I (or pharmaceutically acceptable salts thereof) as an active ingredient, a pharmaceutically acceptable carrier and optionally other therapeutic ingredients or adjuvants. Such additional therapeutic ingredients include, for example, i) opiate agonists or antagonists, ii) calcium channel antagonists, iii) 5HT receptor agonists or antagonists iv) sodium channel antagonists, v) NMDA receptor agonists or antagonists, vi) COX-2 selective inhibitors, vii) NK1 antagonists, viii) non-steroidal anti-inflammatory drugs (“NSAID”), ix) GABA-A receptor modulators, x) dopamine agonists or antagonists, xi) selective serotonin reuptake inhibitors (“SSRI”) and/or selective serotonin and norepinephrine reuptake inhibitors (“SSNRI”), xii) tricyclic antidepressant drugs, xiv) norepinephrine modulators, xv) L-DOPA, xvi) buspirone, xvii) lithium, xviii) valproate, ixx) neurontin (gabapentin), xx) olanzapine, xxi) nicotinic agonists or antagonists including nicotine, xxii) muscarinic agonists or antagonists, xxiii) heroin substituting drugs such as methadone, levo-alpha-acetylmethadol, buprenorphine and naltrexone, and xxiv) disulfiram and acamprosate. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Creams, ointments, jellies, solutions, or suspensions containing the compound of Formula I can be employed for topical use. Mouth washes and gargles are included within the scope of topical use for the purposes of this invention.

Dosage levels from about 0.01 mg/kg to about 140 mg/kg of body weight per day are useful in the treatment of psychiatric and mood disorders such as, for example, schizophrenia, anxiety, depression, panic, bipolar disorders, and circadian disorders, as well as being useful in the treatment of pain which are responsive to calcium channel modulation, or alternatively about 0.5 mg to about 7 g per patient per day. For example, schizophrenia, anxiety, depression, and panic may be effectively treated by the administration of from about 0.01 mg to 75 mg of the compound per kilogram of body weight per day, or alternatively about 0.5 mg to about 3.5 g per patient per day. Pain may-be effectively treated by the administration of from about 0.01 mg to 125 mg of the compound per kilogram of body weight per day, or alternatively about 0.5 mg to about 5.5 g per patient per day. Further, it is understood that the calcium channel modulating compounds of this invention can be administered at prophylactically effective dosage levels to prevent the above-recited conditions.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 1000 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the compounds used represented by Formula L or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions used in the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions used in this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient. Thus, a tablet, cachet, or capsule conveniently contains 0.1 mg, 1 mg, 5 mg, 25 mg, 50 mg, 100 mg, 200 mg, 300 mg, 400 mg, or 500 mg of the active ingredient taken one or two tablets, cachets, or capsules, once, twice, or three times daily.

Pharmaceutical compositions used in the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions used in the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions used in the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions used in this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The compounds and pharmaceutical compositions used in this invention have been found to exhibit biological activity as calcium channel ligands. Accordingly, another aspect of the invention is the treatment in mammals of, for example, schizophrenia, anxiety, depression, panic, bipolar disorders, circadian rhythm and sleep disorders, pain, Parkinson's disease, cognitive dysfunction, epilepsy, drug addiction, drug abuse and drug withdrawal—maladies that are amenable to amelioration through modulation of the calcium channel—by the administration of an effective amount of the compounds of this invention. The term “mammals” includes humans, as well as other animals such as, for example, dogs, cats, horses, pigs, and cattle. Accordingly, it is understood that the treatment of mammals other than humans is the treatment of clinical correlating afflictions to those above recited examples that are human afflictions.

Further, as described above, the compound used in this invention can be utilized in combination with other therapeutic compounds. In particular, the combinations of the clacium channel modulating compound used in this invention can be advantageously used in combination with i) opiate agonists or antagonists, ii) mGluR5 antagonists, iii) 5 HT receptor agonists or antagonists iv) sodium channel antagonists, v) NMDA receptor agonists or antagonists, vi) COX-2 selective inhibitors, vii) NK1 antagonists, viii) non-steroidal anti-inflammatory drugs (“NSAID”), ix) GABA-A receptor modulators, x) dopamine agonists or antagonists, xi) selective serotonin reuptake inhibitors (“SSRI”) and/or selective serotonin and norepinephrine reuptake inhibitors (“SSNRI”), xii) tricyclic antidepressant drugs, xiii) norepinephrine modulators, xiv) L-DOPA, xv) buspirone, xvi) lithium, xvii) valproate, xviii) neurontin (gabapentin), xix) olanzapine, xx) nicotinic agonists or antagonists including nicotine, xxi) muscarinic agonists or antagonists, xxii) heroin substituting drugs such as methadone, levo-alpha-acetylmethadol, buprenorphine and naltrexone, and xxiii) disulfiram and acamprosate.

The abbreviations used herein have the following tabulated meanings. Abbreviations not tabulated below have their meanings as commonly used unless specifically stated otherwise.

| | |
|---|---|
| Ac | Acetyl |
| AIBN | 2,2′-azobis(isobutyronitrile) |
| BINAP | 1,1′-bi-2-naphthol |
| Bn | Benzyl |
| CAMP | cyclic adenosine-3′,5′-monophosphate |
| DAST | (diethylamino)sulfur trifluoride |
| DEAD | diethyl azodicarboxylate |
| DBU | l,8-diazabicyclo[5.4.0]undec-7-ene |
| DIBAL | diisobutylaluminum hydride |
| DMAP | 4-(dimethylamino)pyridine |
| DMF | N,N-dimethylformamide |
| Dppf | 1,1′-bis(diphenylphosphino)-ferrocene |
| EDCI | 1-(3-dimethylaminopropyl)-3-ethylcarbodiimide |
| | hydrochloride |
| Et₃N | Triethylamine |
| GST | glutathione transferase |
| HMDS | Hexamethyldisilazide |
| LDA | lithium diisopropylamide |
| m-CPBA | metachloroperbenzoic acid |
| MMPP | monoperoxyphthalic acid |
| MPPM | monoperoxyphthalic acid, magnesium salt 6H₂O |
| Ms | methanesulfonyl = mesyl = SO₂Me |
| Ms0 | methanesulfonate = mesylate |
| NBS | N-bromo succinimide |
| NSAID | non-steroidal anti-inflammatory drug |
| o-Tol | ortho-tolyl |
| OXONE ® | 2KHSO₅•KHSO₄•K₂SO₄ |
| PCC | pyridinium chlorochromate |
| Pd₂(dba)₃ | Bis(dibenzylideneacetone) palladium(0) |
| PDC | pyridinium dichromate |
| PDE | Phosphodiesterase |
| Ph | Phenyl |
| Phe | Benzenediyl |
| PMB | Para-methoxybenzyl |
| Pye | Pyridinediyl |
| r.t. | Room temperature |
| Rac. | Racemic |
| SAM | aminosulfonyl or sulfonamide or SO₂NH₂ |
| SEM | 2-(trimethylsilyl)ethoxymethoxy |
| SPA | scintillation proximity assay |
| TBAF | Tetra-n-butylammonium fluoride |
| Th | 2- or 3-thienyl |
| TFA | trifluoroacetic acid |
| TFAA | trifluoroacetic acid anhydride |
| THF | Tetrahydrofuran |
| Thi | Thiophenediyl |
| TLC | thin layer chromatography |
| TMS-CN | trimethylsilyl cyanide |
| TMSI | trimethylsilyl iodide |
| Tz | 1H (or 2H)-tetrazol-5-yl |
| XANTPHOS | 4,5-Bis-diphenylphosphany1-9,9-dimethy1-9H-xanthene |
| C₃H₅ | Allyl |

Alkyl Group Abbreviations

| | |
|---|---|
| Me = | Methyl |
| Et = | Ethyl |
| n-Pr = | normal propyl |
| i-Pr = | isopropyl |
| n-Bu = | normal butyl |
| i-Bu = | Isobutyl |
| s-Bu = | secondary butyl |
| t-Bu = | tertiary butyl |
| c-Pr = | cyclopropyl |
| c-Bu = | cyclobutyl |
| c-Pen = | cyclopentyl |
| c-Hex = | cyclohexyl |

Assays Demonstrating Biological Activity

The compounds of this invention were tested by the following assays.

Membrane Preparation:

A710 (HEK293 co-expressing □1 b, □2, □3) cultured in T250 flask were harvested and washed once with buffer A (20 mM HEPES 10 mM EDTA pH=7.4). The pellet was homogenized in buffer A using a Polytron for 20 s. After centrifugation for 10 min, the resulting pellet was washed once with the same buffer and twice with buffer B (20 mM HEPES 0.1 mM EDTA pH=7.4). The final pellet was resuspended in the same buffer and aliquoted and stored at −70° C. Protein contents was measured by the Biorad D C method with bovine serum albumin used as standard.

[³H]-GABApentin Binding:

After thawing, the membranes were washed one time with buffer C (50 mM TRIS pH=7.1) and resuspended in ice cold assay buffer (20 mM HEPES pH=7.4), to have a final protein concentration of 50 □g of protein/well. For the competitive binding experiments, the membranes were incubated with 7 nM [³H]-GABApentin for 1 h at rt in the absence or the presence of at least 11 concentrations of the compounds to be tested. The non-specific binding was measured in the presence of 100 □M GABApentin. At the end of the incubation, the suspension was filtered onto 96 well Whatmann GF/B filter plate (Packard) and washed 3 times with ice-cold assay buffer. The plate was dried and 50 □L of microscint 20 (Packard) was added in each well. The plate was sealed and was counted using a Packard Topcount. The plate was counted (2 min) in normal cpm count mode and transforms in DPM with a constant quench correction.

The compounds of this invention displayed efficacy in the above model by IC₅₀ values of less than 10 μM.

Spinal Nerve Ligation Model (Chung Model):

The spinal nerve ligation model of neuropathic pain was used to assess the effects of test compounds on nerve injury-induced tactile allodynia (S. H. Kim and J. M. Chung, Pain 50:355-363(1992)). Male Sprague Dawley rats (175-200 g) received unilateral tight ligation of the left L5/L6 spinal nerves distal to the dorsal root ganglion using 4-0 silk suture. Behavioral nociceptive testing occurred 7-14 days following spinal nerve ligation by placing the rats in chambers on a wire mesh. Rats were tested for tactile allodynia (decreased hindpaw withdrawal threshold to non-noxious punctate stimulation) by applying a series of calibrated von Frey filaments to the plantar aspect of the left hindpaw ipsilateral to the site of nerve injury. The mean 50% ihindpaw withdrawal threshold (g.) was determined using the Dixon “up-down” non-parametric test (Chaplan et al., J. Neurosci. Methods, 53:55-63(1994)). Rats that displayed a pre-drug withdrawal threshold >4 g were not considered allodynic and were excluded from the study. Following determination of pre-drug withdrawal thresholds, rats received either an i.p. or p.o. injection of test compound. The effect of the test compound on tactile allodynia was determined over time by measuring hindpaw withdrawal thresholds 30, 60, 90, 120 min post-injection.

In above model, EXAMPLE 1 produced a 80% effect after i.p. dosing at 60 mg/kg.

α-Arylaminoacids as an Antagonist of Gabapentin

In this assay, compounds are tested to evaluate whether they may reduce pain by mimicking the mechanism of action of gabapentin. In overview, test compounds are administered alone and in combination with phenylglycine. Compounds whose pain reducing ability is diminished by the addition of phenylglycine are regarded as gabapentin mimics.

Materials and Methods

Male Sprague Dawley rats (Harlan, San Diego, Calif.) weighing 200-250 g were used in the experiments at the time of testing. Rats were housed 3 per cage. All rats were maintained on a standard 12 hr light dark cycle, and had free access to food and water. The experimental procedures described in the present study were approved by the Merck Institutional Animal Care and Use Committee and were performed in accordance with The Guide for the Care and Use of Laboratory Animals.

L5/L6 Spinal Nerve Ligation Injury

Rats were anesthetized with isoflurane (4-5% induction, 2-3% maintenance). Using aseptic technique, the left paraspinal muscles were dissected from the spinous processes at the levels of L4A-S2, and the left L5 and L6 spinal nerves were isolated. Each spinal nerve was tightly ligated with a 4-0 silk suture distal to the dorsal root ganglion (Kim and Chung, 1992). Following spinal nerve ligation, the wound was sutured and the skin was closed with veterinarian grade cyanoacrylate. The rats were allowed to recover for 7 days.

Assessment of Mechanical Allodynia

Mechanical allodynia was determined by measuring the paw withdrawal in response to probing with a series of calibrated von Frey filaments. 7-14 days following spinal nerve ligation, rats were placed in individual Plexiglas chambers on an elevated wire mesh where they were allowed to acclimate for 1 hr. Following the acclimation period, rats were tested for tactile allodynia by applying a series of von Frey filaments to the plantar aspect of the left hind paw ipsilateral to the site of nerve injury. The strength of the von Frey stimuli ranged from 0.4 to 15 g. The mean 50% withdrawal threshold (g.) was determined using the Dixon “up-down” method (Chaplan et al., 1994; Dixon, 1968). Rats that displayed a pre-drug withdrawal threshold >4 g. were not considered allodynic and were excluded from the study. Following determination of pre-drug withdrawal thresholds, rats received a subcutaneous injection of Gabapentin (GBP, 100 mg/kg) or vehicle. The effects on tactile allodynia were determined over time by measuring hind paw withdrawal thresholds 30, 60, 90, 120 min post-injection. For the experiments examining the effects of Phenylglycine on the antiallodynic action of GBP, Phenylglycine (20 mg/kg) or vehicle was injected i.p. 30 min after GBP or vehicle injection.

Data Analysis and Statistics

All behavioral experimental groups consisted of 5-7 rats. For all experiments the data were represented as mean±SEM of the response. Statistical analysis of drug effect was performed by comparing post-drug response to pre drug response using a one-way ANOVA with Dunnett's test and a two way ANOVA with Student-Newman-Keuls Method for post hoc comparisons. Data were converted to % antiallodynia by the formula: % antiallodynia=100×(test value−control value)/(15 g−control value). A computer program was used to calculate the dose required producing a 50% inhibition of the allodynic response at the time of maximal effect.

Reagents

The reagents used in the present experiments were (S) phenylglycine, (D) phenylglycine (Merck Research Laboratories) and gabapentin (Sigma Chemical Co., St. Lous, Mo.). Gabapentin was dissolved in 0.9% saline (pH ˜7), both (S) and (D) phenylglycine were dissolved in saline (pH˜5).

The examples that follow are intended as an illustration of certain preferred embodiments of the invention and no limitation of the invention is implied.

Unless specifically stated otherwise, the experimental procedures were performed under the following conditions. All operations were carried out at room or ambient temperature—that is, at a temperature in the range of 18-25° C. Evaporation of solvent was carried out using a rotary evaporator under reduced pressure (600-4000 pascals: 4.5-30 mm Hg) with a bath temperature of up to 60° C. The course of reactions was followed by thin layer chromatography (TLC) and reaction times are given for illustration only. Melting points are uncorrected and ‘d’ indicates decomposition. The melting points given are those obtained for the materials prepared as described. Polymorphism may result in isolation of materials with different melting points in some preparations. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta ([) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. broad; etc. In addition, “Ar” signifies an aromatic signal. Chemical symbols have their usual meanings; the following abbreviations are used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

Methods of Synthesis

Compounds of the present invention can be prepared according to the following methods. The substituents are the same as in Formula I except where defined otherwise.

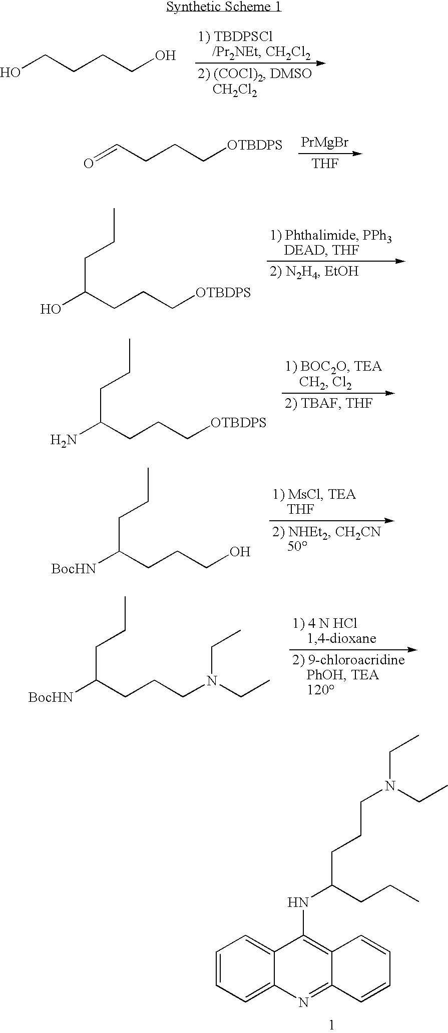

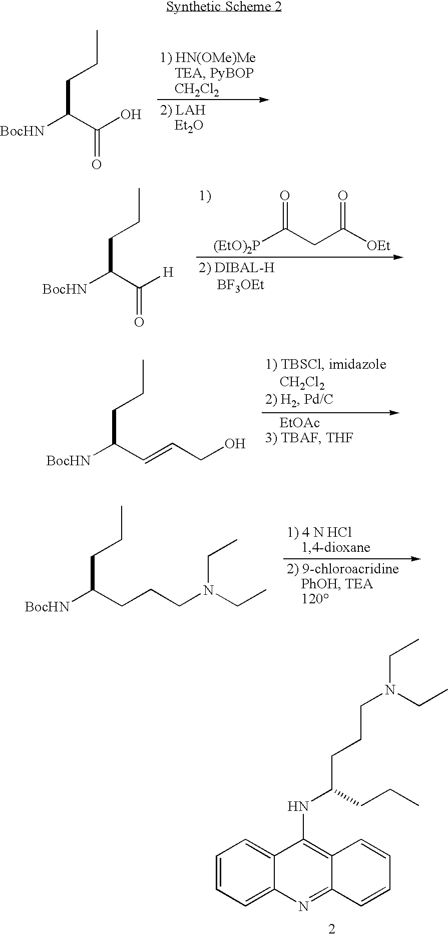

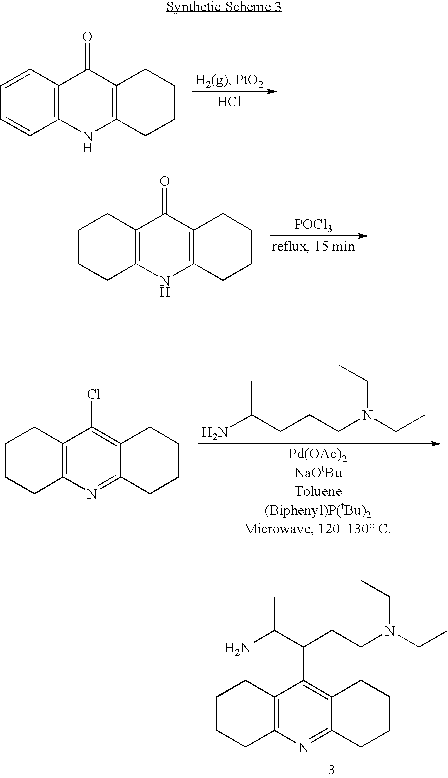

### EXAMPLE 1

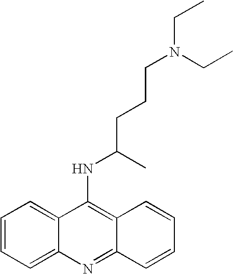 N⁴-Acridin-9-yl-N¹,N¹-diethylpentane-1,4-diamine

A mixture of 9-chloroacridine (7.4 g, 35 mmol), 2-amino-5-diethylaminopentane (5.0 g, 32 mmol), phenol (8.9 g, 95 mmol), and triethylamine (4.8 mL, 35 mmol) was heated to 120 ° C for 1 h, cooled to room temperature, and diluted with CH2Cl2. The resultant mixture was treated with 1 N HCl (100 mL), and then 1 N NaOH (120 mL). The aqueous layer was extracted with CH2Cl2 (×3). The combined organics were washed with brine, dried (Na2SO4), concentrated, and purified by flash chromatography to provide N4-acridin-9-yl-N1,N1-diethylpentane-1,4-diamine as a yellow oil. 1H NMR (CDCl3, 500 MHz) δ 8.10 (m, 4H), 7.68 (m, 2H), 7.39 (m, 2H), 4.81 (br s, 1H), 4.17 (br s, 1), 2.42 (q, 4H), 2.36 (m, 2H), 1.55-1.75 (r, 4H), 1.30 (d, 3H), 0.94 (t, 6H); MS (ESI) 336 (M+H)+.

### EXAMPLE 2

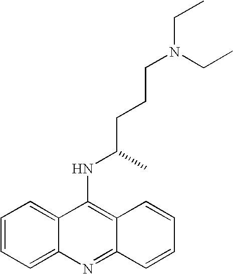 (4S)-N⁴-acridin-9-yl-N¹,N¹-diethylpentane-1,4-diamine

Utilizing the general procedure outlined in the synthesis of N⁴-acridin-9-yl-N¹,N¹-diethylpentane-1,4-diamine, 9-chloroacridine (0.85 g, 4.0 mmol), (S)-2-amino-5-diethylaminopentane (0.50 g, 3.2 mmol), phenol (0.90 g, 9.6 mmol), and triethylamine (0.50 mL, 3.6 mmol) reacted to give (4S)-N⁴-acridin-9-yl-N¹,N¹-diethylpentane-1,4-diamine as a yellow oil.

### EXAMPLE 3

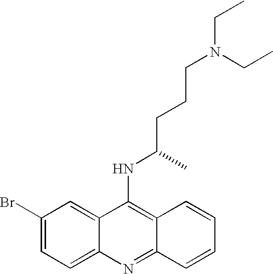 (4S)-N⁴-(2-bromoacridin-9-yl)-N¹,N¹-diethylpentane-1,4-diamine

A mixture of 9(10H)-acridone (0.98 g, 5.0 mmol) and benzyltriethylammonium tribromide (2.3 g, 5.0 mmol) in AcOH (100 mL) was left to stir at room temperature overnight and at 80° C. for 1 h. The mixture was cooled to room temperature, and filtered to provide crude 2-bromo-9(10H)-acridone as a yellow solid.

A mixture of crude 2-bromo-9(10H)-acridone (1.2 g, 4.4 mmol) and DMF (40 μL) in thionyl chloride (6 mL) was heated to 80° C. for 1 h, cooled to room temperature, and concentrated. The residue was poured into NH₄OH solution in ice water, and the aqueous layer was extracted with CHCl₃ (×3). The combined organics were dried (Na2SO4) and concentrated to give crude 2-bromo-9-chloroacridine as a yellow solid. Utilizing the general procedure outlined in the synthesis of N4-acridin-9-yl-N¹,N¹-diethylpentane-1,4-diamine, 2-bromo-9-chloroacridine (0.42 g, 1.4 mmol), (S)-2-amino-5-diethylaminopentane (0.30 g, 1.9 mmol), phenol (0.40 g, 4.3 mmol), and triethylamine (0.50 mL, 3.6 mmol) reacted to give (4S)-N-(2-bromoacridin-9-yl)-N¹,N¹-diethylpentane-1,4-diamine as a yellow oil. ¹H NMR (CDCl₃, 500 MHz) δ 8.28 (s, 1H), 8.11 (d, 2H), 8.00 (d, 1H), 7.73 (m, 2H), 7.45 (t, 1H), 4.92 (br s, 1H), 4.18 (br s, 1H), 2.52 (q, 4H), 2.44 (m, 2H), 1.60-1.82 (m, 4H), 1.34 (d, 3H), 1.00 (t, 6H).

### EXAMPLE 4

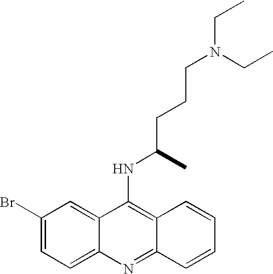 (4R)-N⁴-(2-bromoacridin-9-yl)-N¹,N¹-diethylpentane-1,4-diamine

Utilizing the general procedure outlined in the synthesis of N⁴-acridin-9-yl-N¹,N¹-diethylpentane-1,4-diamine, 2-bromo-9-chloroacridine (0.42 g, 1.4 mmol), (R)-2-amino-5-diethylaminopentane (0.30 g, 1.9 mmol), phenol (0.40 g, 4.3 mmol), and triethylamine (0.50 mL, 3.6 mmol) reacted to give (4R)-N-(2-bromoacridin-9-yl)-N¹,N¹-diethylpentane-1,4-diamine as a yellow oil.

### EXAMPLE 5

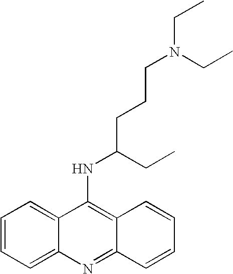 N⁴-acridin-9-yl-N¹,N¹-diethylhexane-1,4-diamine

To a stirred solution of 1,4-butanediol (35 g, 0.39 mol) and diisopropylethylamine (70 mL, 0.40 mol) in CH₂Cl₂ (70 mL) was added TBDPSCl (35 g, 0.13 mol) dropwise over 20 min. The reaction mixture was left to stir overnight, concentrated, and diluted with CH₂Cl₂. The resultant solution was washed with water, dried (MgSO₄), concentrated, and purified by flash chromatography to give 4-{[tert-butyl(diphenyl)silyl]oxy}butan-1-ol as a colorless oil.

To a stirred solution of oxalyl chloride (5.0 mL, 57 mmol) in CH₂Cl₂ (150 mL) was added DMSO (8.5 mL, 120 mmol) in CH₂Cl₂ (20 mL) at −78 ° C. After 15 min at that temperature, a solution of 4-{[tert-butyl(diphenyl)silyl]oxy}butan-1-ol (16 g, 50 mmol) in CH₂Cl₂ (30 mL) was added via cannula. The resultant reaction mixture was stirred at −78° C. for 30 min, treated with triethylamine (35 mL, 250 mmol), and allowed to warm to room temperature as the bath did (1 h). Water was added to the mixture and the aqueous layer was extracted with CH₂Cl₂ (×3). The combined organics were washed with brine, dried (Na₂SO₄), concentrated, and purified by flash chromatography to provide 4-{[tert-butyl(diphenyl)silyl]oxy)butanal as a colorless oil.

To a stirred solution of 4-{[tert-butyl(diphenyl)silyl]oxy}butanal (1.2 g, 3.7 mmol) in THF (20 mL) was added EtMgBr (3.0 M in Et₂O, 3.0 mL, 9.0 mmol). The reaction mixture was left to stir for 2 h, treated with aqueous NH₄Cl solution, and extracted with Et₂O (×3). The combined organics were washed with brine, dried (MgSO₄), concentrated, and purified by flash chromatography to give 6-{[tert-butyl(diphenyl)silyl]oxy}hexan-3-ol as a colorless oil. ¹H NMR (CDCl₃, 500 MHz) δ 7.65-7.67 (m, 4H), 7.37-7.40 (m, 6H), 3.68 (t, 2H), 3.55 (m, 1H), 1,30-1.68 (m, 6H), 1.04 (s, 9H), 0.93 (t, 3H).

To a stirred solution of 6-{[tert-butyl(diphenyl)silyl]oxy}hexan-3-ol (1.4 g, 3.9 mmol), phthalimide (0.57 g, 3.9 mmol), and PPh₃ (1.0 g, 3.9 mmol) in THF (4 mL) was added DEAD (0.62 mL, 3.9 mmol) in THF (4 mL) slowly over 30 min at 0° C. The reaction mixture was left to stir at room temperature overnight, concentrated, and purified by flash chromatography to give 2-(4-{[tert-butyl(diphenyl)silyl]oxy}-1-ethylbutyl)-1H-isoindole-1,3(2H)-dione. ¹H NMR (CDCl₃, 500 MHz) δ 7.35-7.83 (m, 14H), 4.12 (m, 1H), 3.68 (m, 2H), 2.07-2.15 (m, 2H), 1.77-1.92 (m, 2H), 1.47-1.57 (m, 2H), 1.04 (s, 9H), 0.88 (t, 3H).

A mixture of 2-(4-{[tert-butyl(diphenyl)silyl]oxy}-1ethylbutyl)-1H-isoindole-1,3(2H)-dione (1.3 g, 2.7 mmol) and N₂H₄ (0.086 mL, 2.7 mmol) in EtOH (10 mL) was heated to 95° C. overnight, cooled to room temperature, and filtered through fritted glass. The filtrate was concentrated to give crude (4-{[tert-butyl(diphenyl)silyl]oxy}-1-ethylbutyl)amine.

To a stirred solution of crude (4-{[tert-butyl(diphenyl)silyl]oxy}-1-ethylbutyl)amine in CH₂Cl₂ (40 mL) were added triethylamine (3.0 mL, 22 mmol) and BOC₂O (4.0 g, 18 mmol). The reaction mixture was left to stir overnight, treated with 1 N NaOH solution, and left to stir for 10 min. The aqueous layer was extracted with EtOAc (×3). The combined organics were washed with brine, dried (Na₂SO₄), and concentrated to give crude tert-butyl (4-{[tert-butyl(diphenyl)silyl]oxy}-1-ethylbutyl)carbamate. The crude product was taken up into THF (30 mL) and treated with TBAF (1 M in THF, 20 mL, 20 mmol). The reaction mixture was left to stir for 5 h, treated with aqueous NH₄Cl solution, and concentrated. The residue was extracted with EtOAc (×3). The combined organics were washed with brine, dried (Na₂SO₄), concentrated, and purified by flash chromatography to give tert-butyl (1-ethyl-4-hydroxybutyl)carbamate as a colorless oil. ¹H NMR (CDCl₃, 500 MHz) δ 4.37 (br s, 1H), 3.68 (m, 2H), 3.54 (br s, 1H), 1.36-1.66 (m, 6H), 1.46 (s, 9H), 0.93 (t, 3H); MS (ESI) 218 (M+H)⁺.

To a stirred solution of tert-butyl (1-ethyl-4-hydroxybutyl)carbamate (0.19 g, 0.88 mmol) in THF (6 mL) were added triethylamine (0.23 mL, 1.7 mmol) and MsCl (0.10 mL, 1.3 mmol) at −40° C. The reaction mixture was allowed to warm to room temperature as the bath did (40 min) and filtered. The filtrate was concentrated, and the residue was partitioned between brine and CH₂Cl₂. The organic layer was separated, dried (MgSO₄), and concentrated to give crude 4-[(tert-butoxycarbonyl)amino]hexyl methanesulfonate as a colorless oil.

A mixture of crude 4-[(tert-butoxycarbonyl)amino]hexyl methanesulfonate and diethylamine (0.70 mL, 6.8 mmol) in CH₃CN (3 mL) was heated to 50° C. for 2 d, cooled to room temperature, and concentrated to give crude tert-butyl [4-(diethylamino)-1-ethylbutyl]carbamate as a colorless oil. MS (ESI) 273 (M+H)⁺.

Crude tert-butyl [4-diethylamino)-1-ethylbutyl]carbamate was taken up into HCl (4 N in dioxane, 3 mL), and the resultant solution was left to stir for 3 h. The mixture was concentrated to give crude N¹,N¹-diethylhexane-1,4-diamine dihydrochloride. MS (ESI) 173 (M+H)⁺.

Utilizing the general procedure outlined in the synthesis of N⁴-acridin-9-yl-N¹,N¹-diethylpentane-1,4-diamine, 9-chloroacridine (0.20 g, 0.94 mmol), crude N¹,N¹-diethylhexane-1,4-diamine dihydrochloride, phenol (0.25 g, 2.7 mmol), and triethylamine (0.50 mL, 3.6 mmol) reacted to give N⁴-acridin-9-yl-N¹,N¹-diethylhexane-1,4-diamine as a yellow oil. ¹H NMR (CDCl₃, 500 MHz) δ 8.16 (m, 4H), 7.70 (m, 2H), 7.41 (m, 2H), 5.07 (br s, 1H), 4.13 (br s, 1H), 2.47 (q, 4H), 2.38 (m, 2H), 1.55-1.80 (m, 6H), 1.00 (t, 3H), 0.96 (t, 6H); MS (ESI) 350 (M+H)⁺.

### EXAMPLE 6

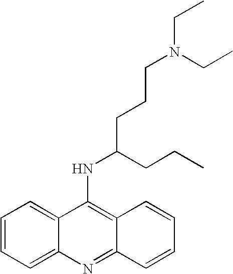 N⁴-acridin-9-yl-N¹,N¹-diethylheptane-1,4-diamine

Utilizing the general procedure outlined in the synthesis of 6-{[tert-butyl(diphenyl)silyl]oxy}hexan-3-ol, 4-{[tert-butyl(diphenyl)silyl]oxy}butanal (1.3 g, 4.0 mmol) and nPrMgBr (2.0 M in Et₂O, 4.5 mL, 9.0 mmol) in THF (20 mL) reacted to give 1-{[tert-butyl(diphenyl)silyl]oxy}heptan-4-ol as a colorless oil.

Utilizing the general procedure outlined in the synthesis of N⁴-acridin-9-yl-N¹,N¹-diethylhexane-1,4-diamine, 9-chloroacridine (0.20 g, 0.94 mmol), crude N¹,N¹-diethylheptane-1,4-diamine dihydrochloride, phenol (0.25 g, 2.7 mmol), and triethylamine (0.50 mL, 3.6 mmol) reacted to give N⁴-acridin-9-yl-N¹,N¹-diethylheptane-1,4-diamine as a yellow oil. ¹H NMR (CDCl₃, 500 MHz) δ 8.20 (d, 2H1), 8.16 (d, 2H), 7.73 (m, 2H), 7.42 (m, 2H), 4.24 (br s, 1H), 2.56 (br s, 4H), 2.48 (br s, 2H), 1.40-1.82 (m, 8H), 1.02 (t, 3H), 0.91 (t, 6H); MS (ESI) 364 (M+H)⁺.

### EXAMPLE 6

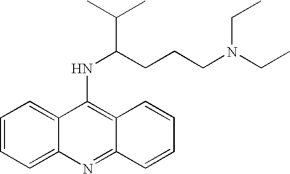 N⁴-acridin-9-yl-N¹,N¹-diethyl-5-methylhexane-1,4-diamine

Utilizing the general procedure outlined in the synthesis 6-{[tert-butyl(diphenyl)silyl]oxy}hexan-3-ol, 4-{[tert-butyl(diphenyl)silyl]oxy}butanal (1.3 g, 4.0 mmol) and iPrMgBr (2.0 M in Et2O, 4.5 mL, 9.0 mmol) in THF (20 mL) reacted to give 6-{[tert-butyl(diphenyl)silyl]oxy}-2-methylhexan-3-ol as a colorless oil.

Utilizing the general procedure outlined in the synthesis of N⁴-acridin-9-yl-N¹,N¹-diethylhexane-1,4-diamine, 9-chloroacridine (0.20 g, 0.94 mmol), crude N¹,N¹-diethyl-5-methylhexane-1,4-diamine dihydrochloride, phenol (0.25 g, 2.7 mmol), and triethylamine (0.50 mL, 3.6 mmol) reacted to give N⁴-acridin-9-yl-N¹,N¹-diethyl-5-methylhexane-1,4-diamine as a yellow oil. ¹H NMR (CDCl₃, 500 MHz) δ 8.21 (d, 2H, 8.17 (d, 2H), 7.72 (m, 2H), 7.42 (m, 2H), 4.18 (br s, 1H), 2.47-2.58 (m, 6H), 2.09 (m, 1H), 1.62-1.83 (m, 4H), 0.99-1.05 (m, 12H); MS (ESI) 364 (M+H)⁺.

### EXAMPLE 7

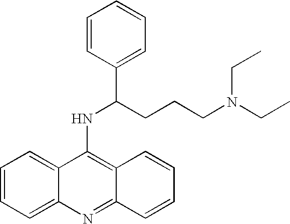 N¹-acridin-9-yl-N⁴,N⁴-diethyl-1-phenylbutane-1,4-diamine

Utilizing the general procedure outlined in the synthesis of 6-{[tert-butyl(diphenyl)silyl]oxy}hexan-3-ol, 4-{[tert-butyl(diphenyl)silyl]oxy}butanal (1.1 g, 3.4 mmol) and PhMgBr (3.0 M in Et2O, 3.0 mL, 9.0 mmol) in THF (20 mL) reacted to give 4-{[tert-butyl(diphenyl)silyl]oxy}-1-phenylbutan-1-ol as a colorless oil.

Utilizing the general procedure outlined in the synthesis of N⁴-acridin-9-yl-N¹,N¹-diethylhexane-1,4-diamine, 9-chloroacridine (0.20 g, 0.94 mmol), crude N⁴,N⁴-diethyl-1-phenylbutane-1,4-diamine dihydrochloride, phenol (0.25 g, 2.7 mmol), and triethylamine (0.50 mL, 3.6 mmol) reacted to give N¹-acridin-9-yl-N⁴,N⁴-diethyl-1-phenylbutane-1,4-diamine as a yellow oil. ¹H NMR (CDCl₃, 500 MHz) δ 8.13 (m, 4H), 7.68 (m, 2H), 7.26-7.42 (m, 7H), 5.14 (t, 1H), 2.47-2.56 (m, 6H), 2.21 (br s, 1H), 2.08 (m, 1H), 1.65 (br s, 1H), 1.54 (br s, 1H), 0.99 (t, 6H); MS (ESI) 398 (M+H)⁺.

### EXAMPLE 8

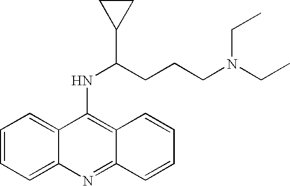 N¹-acridin-9-yl-1-cyclopropyl-N⁴,N⁴-diethylbutane-1,4-diamine

Utilizing the general procedure outlined in the synthesis of 6-{[tert-butyl(diphenyl)silyl]oxy}hexan-3-ol, 4-{[tert-butyl(diphenyl)silyl]oxy}butanal (1.4 g, 4.3 mmol) and clclopropylmagnesium bromide (12 mmol) in THF (20 mL) reacted to give 4-{[tert-butyl(diphenyl)silyl]oxy}-1-cyclopropylbutan-1-ol as a colorless oil.

Utilizing the general procedure outlined in the synthesis of N⁴-acridin-9-yl-N¹,N¹-diethylhexane-1,4-diamine, 9-chloroacridine (0.20 g, 0.94 mmol), crude 1-cyclopropyl-N⁴,N⁴-diethylbutane-1,4-diamine dihydrochloride, phenol (0.25 g, 2.7 mmol), and triethylamine (0.50 mL, 3.6 mmol) reacted to give N¹-acridin-9-yl-1-cyclopropyl-N⁴,N⁴-diethylbutane-1,4-diamine as a yellow oil. ¹H NMR (CDCl₃, 500 MHz) δ 8.16 (m, 4H), 7.72 (m, 2H), 7.43 (m, 2H), 4.99 (br s, 1H), 3.58 (br s, 1H), 2.47-2.60 (m, 6H), 1.75-1.92 (m, 4H), 1.03 (m, 7H), 0.52 (m, 1H), 0.28 (m, 2H), −0.12 (br s, 1H); MS (ESI) 362 (M+H)⁺.

### EXAMPLE 9

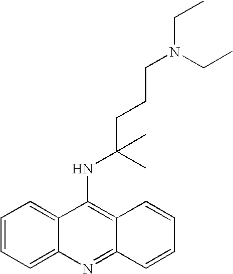 N⁴-acridin-9-yl-N¹,N¹-diethyl-4-methylpentane-1,4-diamine

To a stirred solution of 5-diethylamino-2-pentanone (4.0 g, 25 mmol) in THF (150 mL) was added MeLi (1.6 M in Et₂O, 19 mL, 30 mmol) at −78° C. The reaction mixture was warmed to room temperature as the bath did (1 h), and left to stir overnight. The mixture was treated with aqueous NH₄Cl solution, concentrated, basified with 1 N NaOH solution, and extracted with EtOAc (×3). The combined organics were washed with brine, dried (Na₂SO₄), and concentrated to give crude 5-(diethylamino)-2-methylpentan-2-ol.

Crude 5-(diethylamino)-2-methylpentan-2-ol (1.3 g, 7.7 mmol) was added portionwise to a solution of CH₃CN (0.66 mL, 13 mmol), AcOH (11 mL), and conc. H₂SO₄ (3.6 mL) cooled in an ice bath. The mixture was stirred at room temperature for 4 h, poured into ice water (20 mL), and basified with solid Na₂CO₃. The mixture was extracted with CH₂Cl₂ (×3). The combined organics were washed with brine, dried (Na₂SO₄), and concentrated. The residue was treated with water (7.5 mL), AcOH (7.5 mL), and 12 M HCl (15 mL). The resultant reaction mixture was heated to reflux for 1.5 d, and basified with solid Na₂CO₃, then 1 N NaOH solution. The mixture was extracted with CH₂Cl₂ (×3). The combined organics were washed with brine, dried (Na₂SO₄), and concentrated to give crude N¹,N¹-diethyl-4-methylpentane-1,4-diamine.

Utilizing the general procedure outlined in the synthesis of N⁴-acridin-9-yl-N¹,N¹-diethylpentane-1,4-diamine, 9-chloroacridine (0.27 g, 1.3 mmol), crude N¹,N¹-diethyl-4methylpentane-1,4-diamine (0.20 g, 1.2 mmol), phenol (0.34 g, 3.6 mmol), and triethylamine (0.50 mL, 3.6 mmol) reacted to give N⁴-acridin-9-yl-N¹,N¹-diethyl-4-methylpentane-1,4-diamine as a yellow oil. ¹H NMR (CDCl₃, 500 MHz) δ 8.29 (m, 2H), 8.14 (m, 2H), 7.70 (m, 2H), 7.44 (m, 2H), 4.41 (br s, 1H), 2.55 (q, 4H), 2.47 (m, 2H), 1.72-1.78 (m, 4H), 1.16 (s, 6H), 1.03 (t, 6H); MS (ESI) 350 (M+H)⁺.

### EXAMPLE 10

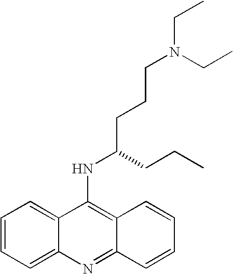 (4S)-N⁴-acridin-9-yl-N¹,N¹-diethylheptane-1,4-diamine

To a stirred solution of N-BOC-L-Norvaline (25 g, 0.12 mol) in CH₂Cl₂ (300 mL) were added triethylamine (47 mL, 0.34 mol), N,O-dimethylhydroxylamine hydrochloride (14 g, 0.14 mol), and PyBOP (60 g, 0.12 mol). The reaction mixture was left to stir for 1 d, treated with 1 N NaOH solution, and left to stir for 1 h. The aqueous layer was extracted with EtOAc (×3). The combined organics were washed with brine, dried (Na₂SO₄), concentrated, and purified by flash chromatography to give N²-(tert-butoxycarbonyl)-N¹-methoxy-N¹-methyl-L-norvalinamide. ¹H NMR (CDCl₃, 500 MHz) δ 5.15 (br s, 1H), 4.70 (br s, 1H), 3.80 (s, 3H), 3.23 (s, 3H), 1.40-1.80 (m, 4H), 1.46 (s, 9H), 0.95 (t, 3H).

To a stirred slurry of LAH (4.7 g, 0.12 mol) in Et₂O (200 mL) at 0° C. was added N²-(tert-butoxycarbonyl)-N¹-methoxy-N¹-methyl-L-norvalinamide (29 g, 0.11 mol) in Et₂O (200 mL) via dropping funnel slowly over 1.5 h. The resultant mixture was left to stir at 0° C. for 30 min, and treated with EtOAc (67 mL) and aqueous KHSO₄ (5%) dropwise at 0° C. The mixture was washed with 1 N HCl (3×100 mL), aqueous NaHCO₃ solution (3×100 mL), and brine (100 mL). The organic layer was dried (MgSO₄), and concentrated to give crude tert-butyl [(1S)-1-formylbutyl]carbamate as a colorless oil.

To a stirred solution of triethyl phosphonoacetate (20 mL, 0.10 mol) in THF (900 mL) was added NaHMDS (1.0 M in THF, 100 mL, 0.10 mol) at −78° C. The resultant mixture was, left to stir for 20 min, treated with crude tert-butyl [(1S)-1-formylbutyl]carbamate (22 g, 0.11 mol) in THF (100 mL) via cannula, and left to stir at −78° C. for 1 h and at 0° C. for 20 min. The reaction mixture was treated with aqueous NH₄Cl solution, and concentrated. The residue was extracted with EtOAc (×3). The combined organics were washed with brine, dried (Na₂SO₄), concentrated, and purified by flash chromatography to give ethyl (2E,4S)-4-[(tert-butoxycarbonyl)amino]hept-2-enoate. ¹H NMR (CDCl₃, 500 MHz) δ 6.85 (dd, 1H), 5.93 (d, 1H), 4.21 (q, 2H), 1.38-1.60 (m, 4H), 1.46 (s, 9H), 1.31 (t, 3H), 0.95 (t, 3H).

To a stirred solution of ethyl (2E,4S)-4-[(tert-butoxycarbonyl)amino]hept-2-enoate (20 g, 73 mmol) in CH₂Cl₂ (400 mL) at −78° C. was added BF₃.OEt₂ (9.3 mL, 73 mmol). The mixture was allowed to stir at that temperature for 30 min before the addition of DIBAL-H (1.0 M in hexanes, 220 mL, 220 mmol). The resultant mixture was left to stir at −78° C. for 1 h, treated with AcOH (42 mL), warmed to room temperature, treated with 10% potassium sodium tartrate solution, and left to stir overnight. The aqueous layer was extracted with EtOAc (×3). The combined organics were washed with aqueous NaHCO₃ solution (×2) and brine, dried (MgSO₄), concentrated, and purified by flash chromatography to give tert-butyl [(1S,2E)-4-hydroxy-1-propylbut-2-en-1-yl]carbamate as a white solid. ¹H NMR (CDCl₃, 500 MHz) δ 5.77 (dt, 1H), 5.63 (dd, 1H), 4.50 (br s, 1H), 4.15 (m, 3H), 1.35-1.50 (m, 4H), 1.45 (s, 9H), 0.93 (t, 3H).

To a stirred solution of tert-butyl [(1S,2E)-4-hydroxy-1-propylbut-2-en-1-yl]carbamate (11 g, 49 mmol) in CH₂Cl₂ (90 mL) was added imidazole (13 g, 190 mmol) followed by TBSCl (14 g, 93 mmol). The reaction mixture was left to stir for 45 min, and treated with water. The aqueous layer was extracted with CH₂Cl₂ (×3). The combined organics were dried (Na₂SO₄), concentrated, and purified by flash chromatography to give tert-butyl ((1S,2E)4-{[tert-butyl(dimethyl)silyl]oxy}-1-propylbut-2-en-1-yl)carbamate.

To a stirred solution of tert-butyl ((1S,2E)-4-{[tert-butyl(dimethyl)silyl]oxy}-1-propylbut-2-en-1-yl)carbamate in EtOAc (200 mL) was added 10% Pd/C. H₂ gas was bubbled through the solution for 1.5 h, and then N₂ gas was bubbled through the solution for 10 min. The mixture was filtered through a pad of SiO₂, and the filtrate was concentrated to give crude tert-butyl ((1S)-4-{[tert-butyl(dimethyl)silyl]oxy}-1-propylbutyl)carbamate.

To a stirred solution of crude tert-butyl ((1S)4-{[tert-butyl(dimethyl)silyl]oxy}-1-propylbutyl)carbamate in THF (400 mL) was added TBAF (1.0 M in THF, 70 mL, 70 mmol) at 0° C. The reaction mixture was left to stir at room temperature for 1 h, treated with aqueous NH₄Cl solution, and concentrated. The residue was extracted with CH₂Cl₂ (×3). The combined organics were washed with brine, dried (Na₂SO₄), concentrated, and purified by flash chromatography to give tert-butyl [(1S)-4-hydroxy-1-propylbutyl]carbamate as a colorless oil.

Utilizing the general procedure outlined in the synthesis of N⁴-acridin-9-yl-N¹,N¹-diethylhexane-1,4-diamine, 9-chloroacridine (1.0 g, 4.7 mmol), crude (4S)-N¹,N¹-diethylheptane-1,4-diamine dihydrochloride (1.1 g, 4.2 mmol), phenol (1.2 g, 13 mmol), and triethylamine (2.0 mL, 14 mmol) reacted to give (S)-N⁴-acridin-9-yl-N¹,N¹-diethylheptane-1,4-diamine as a yellow oil.

### EXAMPLE 11

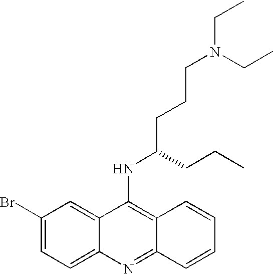 (4S)-N⁴-(2-bromoacridin-9-yl)-N¹,N¹-diethylheptane-1,4-diamine

Utilizing the general procedure outlined in the synthesis of N⁴-acridin-9-yl-N¹,N¹-diethylpentane-1,4-diamine, 2-bromo-9-chloroacridine (0.27 g, 0.92 mmol), crude (4S)-N¹,N¹-diethylheptane-1,4-diamine dihydrochloride (0.30 g, 1.2 mmol), phenol (0.26 g, 2.8 mmol), and triethylamine (0.70 mL, 5.0 mmol) reacted to give (4S)-N⁴-(2-bromoacridin-9-yl)-N¹,N¹-diethylheptane-1,4-diamine as a yellow oil. ¹H NMR (CDCl₃, 500 MHz) δ 8.27 (m, 1H), 8.09 (m, 2H), 7.97 (m, 1H), 7.71 (m, 2H), 7.42 (m, 1H), 5.03 (br s, 1H), 4.13 (m, 1H), 2.38-2.50 (m, 6H), 1.39-1.80 (m, 8H), 0.97 (t, 6H), 0.90 (t, 3H); MS (ESI) 442 (M+H)⁺.

The following tritiated counterpart of Example 10 can be prepared from Example 11.

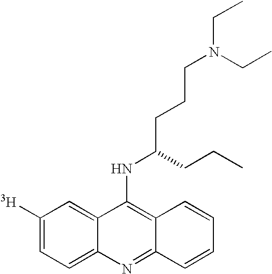

### EXAMPLE 12

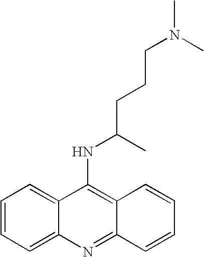 N⁴-Acridin-9-yl-N¹,N¹-dimethylpentane-1,4-diamine

A solution of 5-methyl-2-pyrrolidinone (20 g, 0.20 mol) in 6 N HCl (250 mL) was heated to reflux overnight, and concentrated to give crude 4-aminopentanoic acid. The crude product was taken up into 1 N NaOH (440 mL)/THF (440 mL), and treated with (BOC)₂O (70 g, 0.32 mol). The reaction mixture was left to stir overnight, and concentrated. The residue was washed with Et₂O (×3), acidified with 1 N KHSO₄ solution, and extracted with CH₂Cl₂ (×3). The combined organics were dried (Na₂SO₄), and concentrated to give crude 4-[(tert-butoxycarbonyl)amino]pentanoic acid as a white solid. ¹H NMR (CDCl₃, 500 MHz) δ 4.43 (br s, 1H), 3.74 (br s, 1H), 2.44 (t, 2H), 1.84 (m, 1H), 1.72 (m, 1H), 1.47 (s, 9H), 1.18 (d, 3H).

To a stirred solution of crude 4-[(tert-butoxycarbonyl)amino]pentanoic acid (8.1 g, 37 mmol) in THF (180 mL) at −10° C. was added N-methylmorpholine (4.5 mL, 41 mmol) followed by ethyl chloroformate (3.9 mL, 41 mmol). After 10 min, NaBH₄ (4.2 g, 110 mmol) was added in one portion. MeOH (360 mL) was then added slowly to the reaction mixture over a period of 20 min at 0° C. The solution was stirred for an additional 20 min and then treated with aqueous NH₄Cl solution. The aqueous layer was extracted with EtOAc (×3). The combined organics were washed with brine, dried (Na₂SO₄), concentrated, and purified by flash chromatography to give tert-butyl (4-hydroxy-1-methylbutyl)carbamate as a colorless oil. ¹H NMR (CDCl₃, 500 MHz) δ 4.36 (br s, 1H), 3.67 (br s, 1H), 3.65 (t, 2H), 1.59 (m, 2H), 1.46 (m, 2H), 1.42 (s, 9H), 1.11 (d, 3H).

Utilizing the general procedure outlined in the synthesis of tert-butyl [4-(diethylamino)-1-ethylbutyl]carbamate, 4-[(tert-butoxycarbonyl)amino]pentyl methanesulfonate (0.25 g, 0.89 mmol) and dimethylamine (2.0 M in THF, 3.0 mL, 6.0 mmol) reacted to give crude tert-butyl [4-(dimethylamino)-1-methylbutyl]carbamate as a colorless oil.

Utilizing the general procedure outlined in the synthesis of N⁴-acridin-9-yl-N¹,N¹-diethylhexane-1,4-diamine, 9-chloroacridine (0.23 g, 1.1 mmol), crude N¹,N¹-dimethylpentane-1,4-diamine dihydrochloride, phenol (0.30 g, 3.2 mmol), and triethylamine (0.44 mL, 3.2 mmol) reacted to give N⁴-acridin-9-yl-N¹,N¹-dimethylpentane-1,4-diamine as a yellow oil. ¹H NMR (CDCl₃, 500 MHz) δ 8.13 (m, 4H), 7.70 (m, 2H), 7.40 (m, 2H), 4.23 (m, 1H), 2.26 (m, 2H), 2.17 (s, 6H), 1.76 (m, 2H), 1.64 (m, 2H), 1.33 (d, 3H).

### EXAMPLE 13

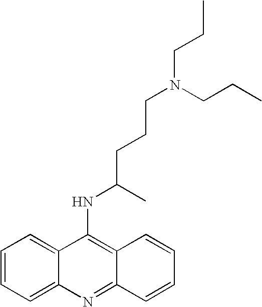 N⁴-Acridin-9-yl-N¹,N¹-dipropylpentane-1,4-diamine

Utilizing the general procedure outlined in the synthesis of tert-butyl [4-(diethylamino)-1-ethylbutyl]carbamate, 4-[(tert-butoxycarbonyl)amino]pentyl methanesulfonate (0.19 g, 0.68 mmol) and dipropylamine (0.46 mL, 3.4 mmol) in CH₃CN (3 mL) reacted to give crude tert-butyl [4-(dipropylamino)-1-methylbutyl]carbamate as a colorless oil.

Utilizing the general procedure outlined in the synthesis of N⁴-acridin-9-yl-N¹,N¹-diethylhexane-1,4-diamine, 9-chloroacridine (0.14 g, 0.68 mmol), crude N¹,N¹-dipropylpentane-1,4-diamine dihydrochloride (0.68 mmol), phenol (0.34 g, 3.6 mmol), and triethylamine (0.50 mL, 3.6 mmol) reacted to give N⁴-acridin-9-yl-N¹,N¹-dipropylpentane-1,4-diamine as a yellow oil. ¹H NMR (CDCl₃, 500 MHz) δ 8.10 (m, 4H), 7.68 (m, 2H), 7.39 (m, 2H), 4.17 (m, 1H), 2.35 (t, 2H), 2.28 (t, 4H), 1.53-1.77 (m, 4H), 1.36 (m, 4H), 1.32 (d, 3H), 0.79 (t, 6H); MS (ESI) 364 (M+H)⁺.

### EXAMPLE 14

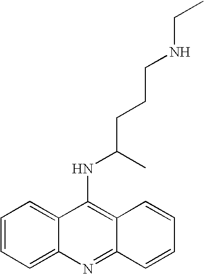 N⁴-Acridin-9-yl-N¹-ethylpentane-1,4-diamine

Utilizing the general procedure outlined in the synthesis of tert-butyl [4-(diethylamino)-1-ethylbutyl]carbamate, 4-[(tert-butoxycarbonyl)amino]pentyl methanesulfonate (0.74 mmol) and ethylamine (2.0 M in THF, 3.0 mL, 6.0 mmol) reacted to give crude tert-butyl [4-(ethylamino)-1-methylbutyl]carbamate as a colorless oil.

Utilizing the general procedure outlined in the synthesis of N⁴-acridin-9-yl-N¹,N¹-diethylhexane-1,4-diamine, 9-chloroacridine (0.16 g, 0.75 mmol), crude N¹-ethylpentane-1,4-diamine dihydrochloride (0.74 mmol), phenol (0.21 g, 2.2 mmol), and triethylamine (1.0 mL, 7.2 mmol) reacted to give N⁴-acridin-9-yl-N¹-ethylpentane-1,4-diamine as a yellow oil. ¹H NMR (CDCl₃, 500 MHz) δ 8.13 (m, 4H), 7.71 (m, 2H), 7.42 (m, 2H), 4.21 (m, 1H), 2.63 (m, 4H), 1.66-1.87 (m, 4H), 1.33 (d, 3H), 1.11 (t, 3H); MS (ESI) 308 (M+H)⁺.

### EXAMPLE 15

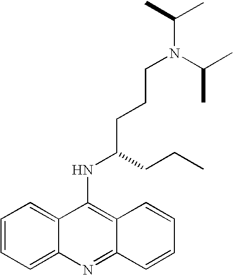 (S)-Acridin-9-yl-[4-(cis-2,6-dimethyl-piperidin-1-yl)-1-propyl-butyl]-amine

Utilizing the general procedure outlined in the synthesis of tert-butyl [4-(diethylamino)-1-ethylbutyl]carbamate, (4S)-4-[(tert-butoxycarbonyl)amino]heptyl methanesulfonate (8.7 mmol), cis-2,6-dimethylpiperidine (2.0 g, 17 mmol), and triethylamine (1.4 mL, 10 mmol) in CH₃CN (9 mL) reacted to give crude tert-butyl [(1S)-4cis-2,6-dimethylpiperidin-1-yl)-1-propylbutyl]carbamate as a colorless oil.

Utilizing the general procedure outlined in the synthesis of N⁴-acridin-9-yl-N¹,N¹-diethyl-hexane-1,4-diamine, 9-chloroacridine (57 mg, 0.75 mmol), [(1S)-4-(cis-2,6-dimethylpiperidin-1-yl)-1-propylbutyl]amine dihydrochloride (50 mg, 0.22 mmol), phenol (72 mg, 0.77 mmol), and triethylamine (0.50 mL, 3.6 mmol) reacted to give N-[(1S)-4-(cis-2,6-dimethylpiperidin-1-yl)-1-propylbutyl]acridin-9-amine as a yellow oil. ¹H NMR (CDCl₃, 500 MHz) δ 8.11 (m, 4H), 7.69 (m, 2H), 7.40 (m, 2H), 4.84 (br s, 1H), 4.15 (br s, 1H), 2.63 (m, 2H), 2.31 (br s, 2H), 1.22-1.75 (m, 14H), 1.00 (d, 3H), 0.96 (d, 3H), 0.92 (t, 3H); MS (ESI) 404 (M+H)⁺.

### EXAMPLE 16

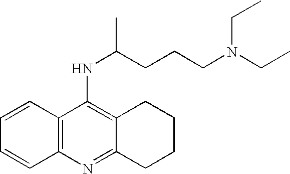 N¹, N¹-Diethyl-N⁴-(1,2,3,4-tetrahydro-acridin-9-yl)-pentane-1,4-diamine

To a solution of 1,2,3,4-tetrahydro-9-acridinone (5.0 g, 25.1 mmol) in thionyl chloride (25 mL) was added DMF (0.2 mL) and the mixture heated to reflux for 1 hour. After cooling, the solution was diluted in CHCl₃ and poured slowly into a vigorously stirred solution of ice (200 g), water (50 mL) and 30% aqueous NH₄OH soln. (50 mL). Stirring was continued for 10 minutes after which the aqueous layer was extracted with CHCl₃ and the combined organic phases dried (MgSO₄), filtered and evaporated to furnish 9chloro-1,2,3,4-tetrahydroacridine as a dark colored foam used without further purification. ¹H NMR (CDCl₃, 500 MHz) δ 8.34 (1H, d), 8.21 (1H, d), 7.78 (1H, m), 7.65 (1H, m), 3.34 (2H, m), 3.04 (2H, m), 1.96 (4H, m).

9-Chloro-1,2,3,4-tetrahydroacridine (762 mg, 3.50 mmol) and phenol (3.12 g, 33.2 mmol) were heated in a re-sealable vessel until homogeneous after which 2-amino-5-diethylaminopentane (1.36 mL, 7.00 mmol) was added and the mixture sealed and heated to 130° C. for 4 hours. After cooling the residue was partitioned between EtOAc and 2M NaOH soln., the organic phase washed with water, dried (MgSO₄), filtered and evaporated to dryness. The resulting oil was purified by chromatography on silica gel eluting with EtOAc:MeOH (0 to 20%) to afford the title compound as a dark oil. ¹H NMR (CDCl₃, 500 MHz) δ 7.90 (1H, dd), 7.54 (1H, m), 7.35 (1H, m), 3.85 (1H, m), 3.74 (1H, br s), 3.07 (2H, m), 2.72 (2H, m), 2.47 (4H, q), 2.39 (2H, m), 1.91 (4H, m), 1.57 (4H, m), 1.17 (3H, d), 0.98 (6H, t); MS (ESI) 340 (M+H)⁺.

### EXAMPLE 17

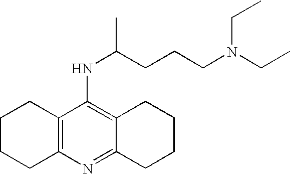 N¹,N¹-Diethyl-N⁴-(1,2,3,4,5,6,7,8-octahydro-acridin-9-yl)-pentane-1,4-diamine

To a mixture of 1,2,3,4-tetrahydro-9-acridinone (5.00 g, 25.1 mmol) in water (75 mL) and conc. HCl (37.5 mL) was added platinum(IV) oxide and the resulting suspension shaken under 40 psi hydrogen gas in a parr apparatus for 16 hours. The resulting solution was filtered through celite then made alkaline with 5M NaOH solution to precipitate 1,3,4,5,6,7,8,10-Octahydro-2H-acridin-9-one.

1,3,4,5,6,7,8,10-Octahydro-2H-acridin-9-one (2.3 g, 11.3 mmol) was added portionwise to phosphorus oxychloride (4 mL) with rapid stirring over a period of 5 minutes then the mixture was heated to reflux for 15 min. After cooling, the solution was diluted in CHCl₃ and poured slowly ice and vigorously stirring was continued for 1 hour. The aqueous layer was neutralized with aq. NH₄OH soln. then extracted with CHCl₃ and the organic phase dried (MgSO₄), filtered and evaporated to furnish 9-Chloro-1,2,3,4,5,6,7,8-octahydro-acridine as a colorless oil which solidified on standing. ¹H NMR (CDCl₃, 500 MHz) δ 2.80 (4H, m), 2.65 (4H, m), 1.76 (8H, m).

In a microwaveable vessel were mixed 9-Chloro-1,2,3,4,5,6,7,8-octahydro-acridine (250 mg, 1.13 mmol), Pd(OAc)₂ (14 mg, 0.062 mmol), sodium tert-butoxide (152 mg, 1.58 mmol), 2-amino-5-diethylaminopentane (0.262 mL, 1.35 mmol), 2-(di-tert-butylphosphino)biphenyl (37 mg, 0.125 mmol) and toluene (2.1 mL). The vessel was then sealed and heated at 120° C. for 20 minutes. The mixture was diluted in EtOAc, washed with water, dried (MgSO₄), filtered and evaporated in vacuo. Chromatographic purification of the residue on silica gel (0 to 10% MeOH in CHCl₃+1% Et₃N) afforded the title compound as a dark oil. ¹H NMR (CDCl₃, 500 MHz) δ 3.47 (1H, m), 3.18 (1H, d), 2.75 (4H, m), 2.43 (8H, m), 2.31 (2H, m), 1.75 (8H, m), 1.42 (3H, m), 1.31 (1H, m), 1.00 (3H, d), 0.94 (6H, t); MS (ESI) 344 (M+H)⁺.

### EXAMPLE 18

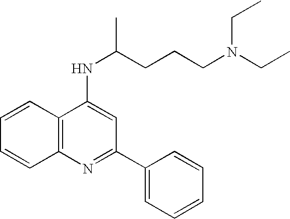 N¹,N¹-Diethyl-N⁴-(2-phenyl-quinolin-4-yl)-pentane-1,4-diamine

A solution of aniline (3.64 mL, 0.04 mol) and ethyl benzoylacetate (6.90 mL, 0.04 mol) in toluene (150 mL) with catalytic p-TsOH was heated to reflux under Dean-Stark conditions for 12 hrs. After cooling, the solution was diluted with EtOAc, washed with sat'd. aq. NaHCO₃, the organic phase dried (MgSO₄), filtered and evaporated in vacuo. The residue was purified by column chromatography on silica gel (0 to 10% EtOAc in heaxnes) to afford ethyl-3-phenyl-3-phenylamino-acrylate as a yellow oil. ¹H NMR (CDCl₃, 500 MHz) δ 10.19 (1H, br s), 7.34 (3H, m), 7.28 (2H, m), 7.07 (2H, m), 6.91 (1H, m), 6.66 (2H, d), 4.99 (1H, s), 4.21 (2H, q), 1.30 (3H, t).

Diphenyl ether (20 mL) was heated to 240° C. and a solution of ethyl-3-phenyl-3-phenylamino-acrylate (1.2 g, 4.49 mmol) in diphenyl ether (5 mL) was added and the yellow solution heated to 250° C. for 10 minutes. After cooling to room temperature, hexanes were added and the resultant precipitate filtered, washed with hexanes and dried to afford 2-phenyl-1H-quinolin-4-one as a colorless solid. ¹H NMR (d₆-DMSO, 500 MHz) δ11.77 (1H, br s), 8.13 (1H, dd), 7.85 (2H, m), 7.78 (1H, d), 7.70 (1H, m), 7.61 (3H, m), 7.36 (1H, m), 6.36 (1H, br s). MS (ESI) 223 (M+H)⁺.

2-phenyl-1H-quinolin-4-one (800 mg, 3.62 mmol) was heated to reflux in phosphorus oxychloride for 15 minutes, allowed to cool the poured in to ice water and vigorously stirred for 1 hour. The resulting solution was neutralized with aq. NH₄OH soln., extracted with CHCl₃ and the organic phase dried (MgSO₄), filtered and evaporated in vacuo to yield 4-chloro-2-phenyl-quinoline as a colorless solid. ¹H NMR (CDCl₃, 500 MHz) δ 8.16 (1H, dd), 8.11 (1H, d), 8.07 (2H, m), 7.90 (1H, s), 7.71 (1H, m), 7.55 (1H, m), 7.47 (2H, m), 7.41 (1H, m).

In a microwaveable vessel were mixed 4-Chloro-2-phenyl-quinoline (300 mg, 1.25 mmol), Pd(OAc)₂ (20 mg, 0.089 mmol), sodium tert-butoxide (168 mg, 1.75 mmol), 2-amino-5-diethylaminopentane (0.315 mL, 1.62 mmol), 2-(di-tert-butylphosphino)biphenyl (54 mg, 0.180 mmol) and toluene (2.5 mL). The vessel was then sealed and heated at 130° C. for 15 minutes. The mixture was diluted in EtOAc, washed with water, dried (MgSO₄), filtered and evaporated in vacuo. Chromatographic purification of the residue on silica gel (0 to 10% MeOH in CHCl₃+1% Et₃N) afforded the title compound as a dark oil which was further purified by preparative HPLC. ¹H NMR (CDCl₃, 500 MHz) δ 8.07 (3H, m), 7.74 (1H, d), 7.64 (1H, m), 7.50 (2H, m), 7.43 (1H, m), 7.40 (1H, m), 6.87 (1H, s), 5.15 (1H, d), 3.83 (1H, m), 2.53 (4H, q), 2.46 (2H, m), 1.76 (1H, m), 1.65 (3H, m), 1.35 (3H, d), 1.00 (6H, t); MS (ESI) 362 (M+H)⁺.

### EXAMPLE 19

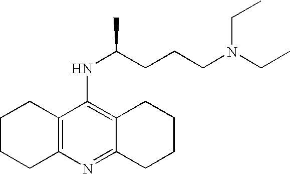 (4S)-N¹,N¹-Diethyl-N⁴-1,2,3,4,5,6,7,8-octahydro-acridin-9-yl)-pentane-1,4-diamine

Prepared in an analogous fashion to that described for N¹,N¹-Diethyl-N⁴-(1,2,3,4,5,6,7,8-octahydro-acridin-9-yl)-pentane-1,4-diamine employing enantiopure (S)-2-amino-5-diethylaminopentane. Data identical to that reported for N¹,N¹-Diethyl-N-(1,2,3,4,5,6,7,8-octahydro-acridin-9-yl)-pentane-1,4-diamine.

Other variations or modifications, which will be obvious to those skilled in the art, are within the scope and teachings of this invention. This invention is not to be limited except as set forth in the following claims.

What is claimed is:

1. A compound of Formula (I): 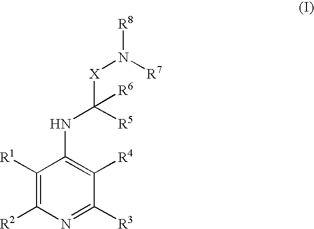
or N-oxide and pharmaceutically acceptable salts thereof, wherein
R¹ is selected from the group consisting of
(a) Hydrogen,
(b) halo,
(c) —C₀₋₆alkyl-aryl,
(d) —C₀₋₆alkyl-heteroaryl,
(e) —C₁₋₆alkyl, optionally substituted with 1, 2 or 3 halo atoms,
(f) —C₀₋₆alkyl-C₃₋₆cycloalkyl, and
(g) -heteroC₀₋₆alkyl;
R² is selected from the group consisting of
(a) Hydrogen,
(b) halo,
(c) —C₀₋₆alkyl-aryl,
(d) —C₀₋₆alkyl-heteroaryl,
(e) —C₁₋₆alkyl, optionally substituted with 1, 2 or 3 halo atoms,
(f) —C₀₋₆alkyl-C₃₋₆cycloalkyl, and
(g) -heteroC₀₋₆alkyl;
or R¹ and R² are joined so that together with the atoms to which they are attached there is formed a saturated or unsaturated ring with 04 heteroatoms, selected from phenyl, said ring optionally mono or di-substituted with sustituents independently selected from hydroxyl, halo, —C₁₋₆alkyl, —O—C₁₋₆alkyl, —NO₂, —CF₃, aryl, heteroaryl, and heteroC₁₋₆alkyl;
R³ is selected from the group consisting of
(a) Hydrogen,
(b) halo,
(c) —C₀₋₆alkyl-aryl,
(d) —C₀₋₆alkyl-heteroaryl,
(e) —C₁₋₆alkyl, optionally substituted with 1, 2 or 3 halo atoms,
(f) —C₀₋₆alkyl-C₃₋₆cycloalkyl, and
(g) -heteroC₀₋₆alkyl;
R⁴ is selected from the group consisting of
(a) Hydrogen,
(b) halo,
(c) —C₀₋₆alkyl-aryl,
(d) —C₀₋₆alkyl-heteroaryl,
(e) —C₁₋₆alkyl, optionally substituted with 1, 2 or 3 halo atoms,
(f) —C₀₋₆alkyl-C₃₋₆cycloalkyl, and
(g) -heteroC₀₋₆alkyl;
or R³ and R⁴ are joined so that together with the atoms to which they are attached there is formed a saturated or unsaturated ring with 0-4 heteroatoms, selected from phenyl, said ring optionally mono or di-substituted with sustituents independently selected from hydroxyl, halo, —C₁₋₆alkyl, —O—C₁₋₆alkyl, —NO₂, —CF₃, aryl, heteroaryl, and heteroC₁₋₆alkyl;
R⁵ is selected from the group consisting of
(a) Hydrogen,
(b) —C₀₋₆alkyl-aryl,
(c) —C₀₋₆alkyl-heteroaryl,
(d) —C₁₋₆alkyl, optionally substituted with 1, 2 or 3 halo atoms,
(e) —C₀₋₆alkyl-C₃₋₆cycloalkyl, and
(f) -heteroC₀₋₆alkyl;
wherein R⁵ choices (b), (c), (d), (e) and (f) are each optionally substituted with a substituent selected from hydroxyl, halo, —NO₂ and CF₃;
R⁶ is selected from the group consisting of
(a) hydrogen,
(b) —C₁₋₃alkyl,
wherein R⁶ choices (b) is optionally substituted with a substituent selected from hydroxyl, halo, —NO₂ and CF₃;
or R ⁵ and R⁶ are joined so that together with the atoms to which they are attached there is formed a saturated or unsaturated ring with 0-4 heteroatoms, selected from phenyl, said ring optionally mono or di-substituted with sustituents independently selected from hydroxyl, halo, —C₁₋₆acyl, —C₁₋₆alkyl, —NO₂, —CF₃, aryl, heteroaryl, and heteroC₁₋₆alkyl;
R⁷ is selected from the group consisting of
(a) Hydrogen,
(b) —C₀₋₃alkyl-aryl,
(c) —C₀₋₃alkyl-heteroaryl,
(d) —C₁₋₆alkyl,
(e) —C₀₋₃alkyl-C₃₋₆cycloalkyl, and
(f) -heteroC₀₋₆alkyl;
wherein R⁷ choices (b), (c), (d), (e) and (f) are each optionally substituted with a substituent selected from hydroxyl, halo, —NO₂ and CF₃;
R⁸ is selected from the group consisting of
(a) Hydrogen,
(b) —C₀₋₃alkyl-aryl,
(c) —C₀₋₃alkyl-heteroaryl,
(d) —C₁₋₆alkyl,
(e) —C₀₋₃alkyl-C₃₋₆cycloalkyl, and
(f) -heteroC₀₋₆alkyl;
wherein R⁸ choices (b), (c), (d), (e) and (f) are each optionally substituted with a substituent selected from hydroxyl, halo, —NO₂ and CF₃;
or R⁶ and R⁸ are joined so that together with the atoms to which they are attached there is formed a saturated or unsaturated ring with 1-4 heteroatoms, selected from phenyl, said ring optionally mono or di-substituted with sustituents independently selected from hydroxyl, halo, —C₁₋₆alkyl, —O—C₁₋₆alkyl, —NO₂, —CF₃, aryl, heteroaryl, and heteroC₁₋₆alkyl;
or R⁷ and R⁸ are joined so that together with the atoms to which they are attached there is formed a saturated or unsaturated ring with 0-4 heteroatoms, selected from phenyl, said ring optionally mono or di-substituted with sustituents independently selected from hydroxyl, halo, —C₁₋₆alkyl, —O—C₁₋₆alkyl, —NO₂, —CF₃, aryl, heteroaryl, and heteroC₁₋₆alkyl;
R⁹ is selected from the group consisting of
(a) C1-6alkyl,
(b) C3-6cycloalkyl,
(c) aryl, and
(d) heteroaryl; and
X is selected from the group consisting of
(a) C₁₋₆alkylene,
(b) O,
(c) S,
(d)S(O)₂,
(e) NR⁹, and
(f) C(O),
with the proviso that either R¹ and R² or R³ and R⁴ must be joined together to form a ring.

2. A compound according to claim 1
R¹ is selected from the group consisting of
(a) hydrogen,
(b) phenyl or naphthyl,
(c) —C₁₋₆alkyl, optionally substituted with 1, 2 or 3 halo atoms,
(d) —O—C₁₋₆alkyl; and
R² is selected from the group consisting of
(a) hydrogen,
(b) phenyl or naphthyl,
(c) —C₁₋₆alkyl, optionally substituted with 1, 2 or 3 halo atoms
(d) —O—C₁₋₆alkyl;
or R¹ and R² are joined so that together with the atoms to which they are attached there is formed a ring selected from phenyl, naphthyl and cyclohexyl, said ring optionally mono or di-substituted with sustituents independently selected from hydroxyl, halo, —C₁₋₆alkyl, —O—C₁₋₆alkyl, —NO₂ and —CF₃.

3. A compound according to claim 2
R¹ and R² are joined so that together with the atoms to which they are attached there is formed a ring selected from phenyl, naphthyl and cyclohexyl, said ring optionally mono or di-substituted with sustituents independently selected from hydroxyl, halo, —C₁₋₆alkyl, —O—C₁₋₆alkyl, —NO₂ and —CF₃.

4. A compound according to claim 1 wherein:
R³ is selected from the group consisting of
(a) hydrogen,
(b) phenyl or naphthyl,
(c) —C₁₋₆alkyl, optionally substituted with 1, 2 or 3 halo atoms
(d) —O—C₁₋₆allyl; and
R⁴ is selected from the group consisting of
(a) hydrogen,
(b) phenyl, naphthyl or pyridyl,
(c) —C₁₋₆alkyl, optionally substituted with 1, 2 or 3 halo atoms,
(d) —O—C₁₋₆alkyl;
or R³ and R⁴ are joined so that together with the atoms to which they are attached there is formed a ring selected from phenyl and cyclohexyl, said ring optionally mono or di-substituted with sustituents independently selected from hydroxyl, halo, —C₁₋₆alkyl, —O—C₁₋₆alkyl, —NO₂ and —CF₃.

5. A compound according to claim 4 wherein:
R³ and R⁴ are joined so that together with the atoms to which they are attached there is formed a ring selected from phenyl and cyclohexyl, said ring optionally mono or di-substituted with sustituents independently selected from hydroxyl, halo, —C₁₋₆alkyl, —O—C₁₋₆alkyl, —NO₂ and —CF₃.

6. A compound according to claim 1 wherein:
R⁵ is selected from the group consisting of
(a) hydrogen,
(b) —C₁₋₃alkyl,
(c) phenyl or naphthyl,
(d) —C₃₋₆cycloalkyl.

7. A compound according to claim 1 wherein:
R⁶ is selected from the group consisting of
(a) hydrogen,
(b) —C₁₋₃alkyl;
R⁷ is selected from the group consisting of
(a) hydrogen,
(b) —C₁₋₆alkyl,
(c) —C₁₋₄alkylphenyl; and
R⁸ is selected from the group consisting of
(a) hydrogen,
(b) —C₁₋₆alkyl;
or R⁶ and R⁸ are joined so that together with the atoms to which they are attached there is formed a piperidine or pyridine or ring, optionally mono- or di-substituted with substituents selected from the group consisting of hydroxyl, —O—C₁₋₆alkl and —C₁₋₆alkyl;
or R⁷ and R⁸ are joined so that together with the atoms to which they are attached there is formed a piperidine, morpholine, pyridine, pyrazole, imidazole or tetrazole ring, optionally mono- or di-substituted with substituents selected from the group consisting of hydroxyl, —OC₁₋₆alkl and —C₁₋₆alkyl.

8. A compound according to claim 1 wherein:
X is CH₂CH₂CH₂.

9. A compound according to claim 1 of Formula II 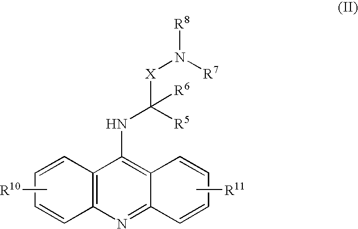
wherein:
R⁵ is selected from the group consisting of
(a) hydrogen,
(b) —C₁₋₃alkyl,
(c) phenyl or naphthyl,
(d) —C₃₋₆cycloalkyl;
R⁶ is
(a) hydrogen,
(b) —C₁₋₃alkyl;
R⁷ is selected from the group consisting of
(a) hydrogen,
(b) —C₁₋₄alkyl,
(c) —C₁₋₂alkylphenyl;
R⁸ is —C₁₋₄alkyl;
R¹⁰ and R¹¹ are each selected from the group consisting of Hydrogen, hydroxyl, halo, —C₁₋₃alkyl, —O—C₁₋₃alkyl, —NO₂ and —CF₃; and X is CH₂CH₂CH₂.

10. A compound according to claim 9 wherein:
R6 is hydrogen.

11. A compound according to claim 10 wherein
R⁵ is selected from the group consisting of —C₁₋₃alkyl, phenyl, naphthyl and —C₃₋₆cycloalkyl.

12. A compound according to claim 1 selected from the group consisting of: 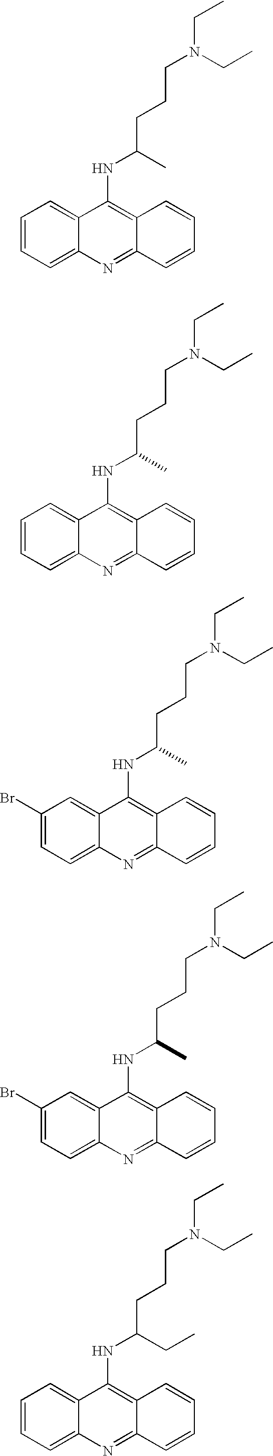 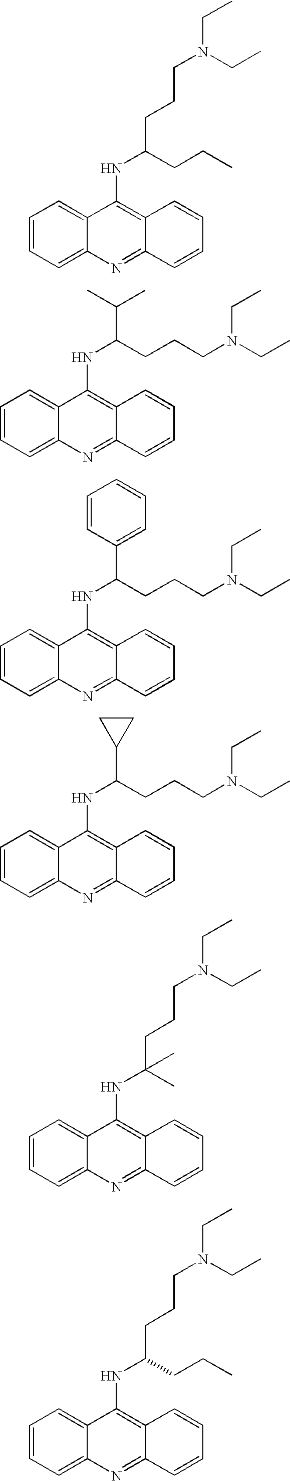 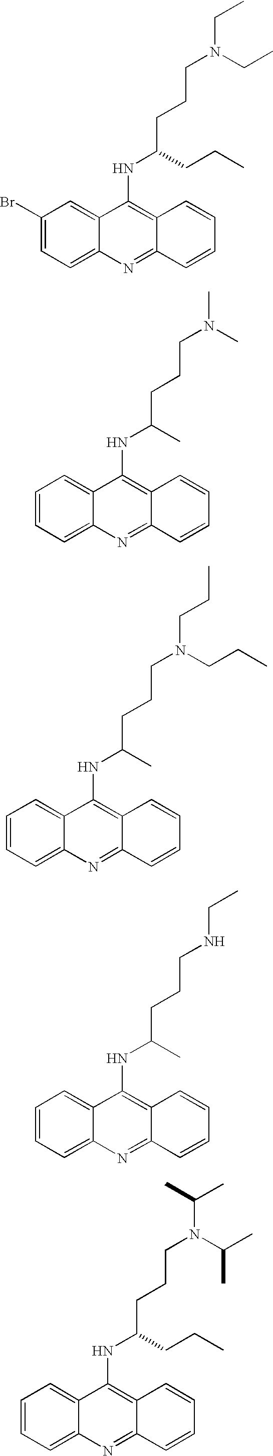 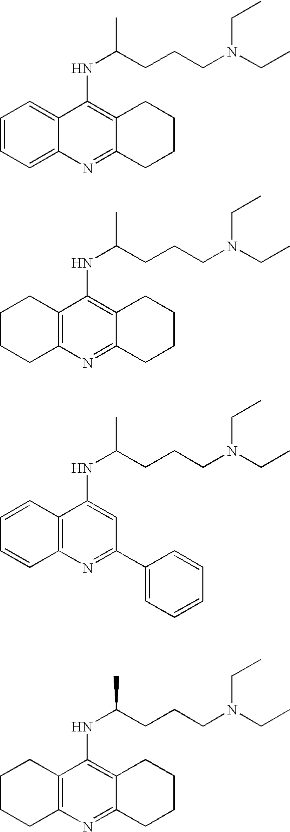
or a pharmaceutically acceptable salt thereof.

13. A pharmaceutical composition for treating an indication mediated by the binding of an a28 subunit of voltage gated calcium channel, comprising a therapeutically effective amount a of a compound according to claim 1 or a pharmaceutically acceptable salt thereof; and a pharmaceutically acceptable acrrier.

14. A composition according to claim 16, said composition further comprising i) an opiate agonist, ii) an opiate antagonist, iii) an mGluR5 antagonist, iv) a 5HT receptor agonist, v) a 5HT receptor antagonist, vi) a sodium channel antagonist, vii) an NMDA receptor agonist, viii) an NMDA receptor antagonist, ix) a COX-2 selective inhibitor, x) an NK1 antagonist, xi) a non-steroidal anti-inflammatory drug, xii) a GABA-A receptor modulator, xiii) a dopamine agonist, xiv) a dopamine antagonist, xv) a selective serotonin reuptake inhibitor, xvi) a tricyclic antidepressant drug, xvii) a norepinephrine modulator, xviii) L-DOPA, xix) buspirone, xx) a lithium salt, xxi) valproate, xxii) neurontin, xxiii) olanzapine, xxiv) a nicotinic agonist, xxv) a nicotinic antagonist, xxvi) a muscarinic agonist, xxvii) a muscarinic antagonist, xxviii) a selective serotonin and norepinephrine reuptake inhibitor (SSNRI), xxix) a heroin substituting drug, xxx) disulfiram, or xxxi) acamprosate.

15. A composition according to claim 1, wherein said heroin substituting drug is methadone, levo-alpha-acetylmethadol, buprenorphine or naltrexone.

16. A method of treatment of neuropathic pain comprising a step of administering an effective amount of a compound according to claim 1.

17. A method of treatment or prevention of pain comprising the step of administering a therapeutically effective amount, or a prophylactically effective amount, of the compound according to claim 1 or a pharmaceutically acceptable salt thereof.

18. A method of treatment or prevention of a pain disorder wherein said pain disorder is acute pain, persistent pain, chronic pain, inflammatory pain, or neuropathic pain, comprising the step of administering a therapeutically effective amount, or a prophylactically effective amount, of the compound according to claim 1 or a pharmaceutically acceptable salt thereof.

19. A method of treatment or prevention of anxiety, depression, bipolar disorder, psychosis, drug withdrawal, tobacco withdrawal, memory loss, cognitive impairment, dementia, Alzheimer's disease, schizophrenia or panic comprising the step of administering a therapeutically effective amount, or a prophylactically effective amount, of the compound according to claim 1 or a pharmaceutically acceptable salt thereof.

20. A method of treatment or prevention of disorders of extrapyramidal motor function comprising the step of administering a therapeutically effective amount, or a prophylactically effective amount, of the compound according to claim 1 or a pharmaceutically acceptable salt thereof.

21. The method of claim 20 wherein said disorder of extrapyramidal motor function is Parkinson's disease, progressive supramuscular palsy, Huntington's disease, Gilles de la Tourette syndrome, or tardive dyskinesia.

22. A method of treatment or prevention of anxiety disorders comprising the step of administering a therapeutically effective amount, or a prophylactically effective amount, of the compound according to claim 1 or a pharmaceutically acceptable salt thereof.

23. A method of claim 22 wherein said anxiety disorder is panic attack, agoraphobia or specific phobias, obsessive-compulsive disorders, post-traumatic stress disorder, acute stress disorder, generalized anxiety disorder, eating disorder, substance-induced anxiety disorder, or nonspecified anxiety disorder.

24. A method of treatment or prevention of neuropathic pain comprising the step of administering a therapeutically effective amount, or a prophylactically effective amount, of the compound according to claim 1 or a pharmaceutically acceptable salt thereof.

25. A method of treatment or prevention of Parkinson's Disease comprising the step of administering a therapeutically effective amount, or a prophylactically effective amount, of the compound according to claim 1 or a pharmaceutically acceptable salt thereof.

26. A method of treatment or prevention of depression comprising the step of administering a therapeutically effective amount, or a prophylactically effective amount, of the compound according to claim 1 or a pharmaceutically acceptable salt thereof.

27. A method of treatment or prevention of epilepsy comprising the step of administering a therapeutically effective amount, or a prophylactically effective amount, of the compound according to claim 1 or a pharmaceutically acceptable salt thereof.

28. A method of treatment or prevention of inflammatory pain comprising the step of administering a therapeutically effective amount, or a prophylactically effective amount, of the compound according to claim 1 or a pharmaceutically acceptable salt thereof.

29. A method of treatment or prevention of cognitive dysfunction comprising the step of administering a therapeutically effective amount, or a prophylactically effective amount, of the compound according to claim 1 or a pharmaceutically acceptable salt thereof.

30. A method of treatment or prevention of drug addiction, drug abuse and drug withdrawal comprising the step of administering a therapeutically effective amount, or a prophylactically effective amount, of the compound according to claim 1 or a pharmaceutically acceptable salt thereof.

31. A method of treatment or prevention of bipolar disorders comprising the step of administering a therapeutically effective amount, or a prophylactically effective amount, of the, compound according to claim 1 or a pharmaceutically acceptable salt thereof.

32. A method of treatment or prevention of circadian rhythm and sleep disorders comprising the step of administering a therapeutically effective amount, or a prophylactically effective amount, of the compound according to claim 1 or a pharmaceutically acceptable salt thereof.

33. The method of claim 32 wherein the circadian rhythm and sleep disorders are shift-work induced sleep disorder or jet-lag.
